(19) United States
(12) Patent Application Publication
(10) Pub. No.: US 2008/0200431 A1
(43) Pub. Date: Aug. 21, 2008

(54) Heterocyclic Sulfonamide Derivatives as Inhibitors of Factor Xa

(75) Inventors: Christer Alstermark, Molndal (SE); Kosrat Amin, Molndal (SE); Kjell Andersson, Molndal (SE); Ulf Fahlander, Molndal (SE); Kenneth Granberg, Molndal (SE); Daniel Hovdal, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/994,844

(22) PCT Filed: Jul. 5, 2006
(86) PCT No.: PCT/SE2006/000840

§ 371 Date: Jan. 7, 2008

(30) Foreign Application Priority Data
Jul. 8, 2005 (SE) 0501621-7

(51) Int. Cl. A61K 31/727; C07D 401/14; A61K 31/501; A61P 7/00; C07D 487/04; A61K 31/496

(52) U.S. Cl. 514/56; 544/238; 514/252.02; 544/362; 514/253.04; 544/357; 514/252.11

(57) ABSTRACT

The invention relates to compounds of formula (I), Chemical formula should be inserted here. Please see paper copy wherein R¹, R², R³ and R⁴ are independently selected from carbon and nitrogen, and where is nitrogen; A¹ is a single bond or a double bond; n is 0, 1, 2 or 3; each R⁵ is independently selected from hydrogen, halogen, C₁₋₃ alkyl, oxo, oxy, oxido and thioxo; R⁶ is hydrogen or oxo; m is 0, 1, 2 or 3; A² is a single bond or a double bond; each R⁷ is independently selected from hydrogen, hydroxy, oxo, C₁₋₅alkyl, carboxy, cyano, tetrazolyl, N—C₁₋₅alkyltetrazolyl, oxazolyl, C₁₋₅oxazolyl, isoxazolyl, C₁₋₅ isoxazolyl, hydroxyC₁₋₅alkyl, carboxy C₁₋₅alkyl, C₁₋₅alkoxyoxo C₁₋₅alkyl, carbamoyl, C₁₋₅alkylcarbamoyl, di(C₁₋₅alkyl)carbamoyl, carbamoyl C₁₋₄alkyl, C₁₋₅alkylcarbamoyl C₁₋₄alkyl, di(C₁₋₅alkyl)carbamoyl C₁₋₄alkyl, hydroxy C₁₋₅alkylcarbamoyl, C₁₋₅alkoxy C₁₋₅alkylcarbamoyl, hydroxy C₁₋₅alkylcarbamoyl C₁₋₄alkyl, C₁₋₅alkoxy C₁₋₅alkylcarbamoyl C₁₋₄alkyl, —CONR⁸(CH₂)ₓS(O)ₚR⁹, —CONH(CH₂)_{q}NR¹⁰R¹¹, —C₁₋₅alkyl-Y¹, —COOCHR¹⁷R¹⁸ and —CONR¹⁷R¹⁸; and R³⁰ is hydrogen, amino, methyl or halogen; or a pharmaceutically acceptable salt thereof, said compounds possess antithrombotic and anticoagulant properties and are accordingly useful in methods of treatment of humans or animals. The invention also relates to processes for the preparation of the compounds, to their use, to pharmaceutical compositions comprising them, to their use in the manufacture of medicaments for use in the production of an antithrombotic or anticoagulant effect, and to combinations comprising them.

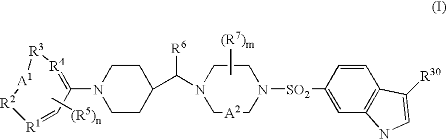

The invention relates to novel heterocyclic derivatives, or pharmaceutically-acceptable salts thereof, which possess antithrombotic and anticoagulant properties and are accordingly useful in methods of treatment of humans or animals. The invention also relates to processes for the preparation of the heterocyclic derivatives, to their use, to pharmaceutical compositions comprising them, to their use in the manufacture of medicaments for use in the production of an antithrombotic or anticoagulant effect, and to combinations comprising them.

The antithrombotic and anticoagulant effect produced by the compounds of the invention is believed to be attributable to their strong inhibitory effect against the activated coagulation protease known as Factor Xa. Factor Xa is one of a cascade of proteases involved in the complex process of blood coagulation. The protease known, as thrombin is the final protease in the cascade and Factor Xa is the preceding protease, which cleaves prothrombin to generate thrombin.

Certain compounds are known to possess Factor Xa inhibitory properties and the field has been reviewed by B.-Y. Zhu, R. M. Scarborough, Current Opinion in Cardiovascular, Pulmonary & Renal Investigational Drugs, 1999, 1(1), 63-88. Thus it is known that two proteins, one known as recombinant antistasin (r-ATS) and the other known as recombinant tick anticoagulant protein (r-TAP), are specific direct Factor Xa inhibitors which possess antithrombotic properties in various animal models of thrombotic disease.

It is also known that certain non-peptidic compounds possess Factor Xa inhibitory properties. Of the low molecular weight inhibitors mentioned in the review by B.-Y. Zhu and R. M. Scarborough, many inhibitors possess a strongly basic group such as an amidinophenyl or amidinonaphthyl group.

We have now found that certain heterocyclic derivatives possess Factor Xa inhibitory activity. Many of the compounds of the present invention also possess the advantage of being selective Factor Xa inhibitors, that is the enzyme Factor Xa is inhibited strongly at concentrations of test compound which do not inhibit or which inhibit to a lesser extent the enzyme thrombin which is also a member of the blood coagulation enzymatic cascade.

The compounds of the present invention possess activity useful in the treatment or prevention of a variety of medical disorders where anticoagulant therapy is indicated, for example in the treatment or prevention of thrombotic conditions such as coronary artery and cerebrovascular disease. Further examples of such medical disorders include various cardiovascular and cerebrovascular conditions such as myocardial infarction, the rupture of atherosclerotic plaques, venous or arterial thrombosis, coagulation syndromes, vascular injury including reocclusion and restenosis following angioplasty and coronary artery bypass surgery, thrombus formation after the application of blood vessel operative techniques or after general surgery such as hip replacement surgery, the introduction of artificial heart valves or on the recirculation of blood, cerebral infarction, cerebral thrombosis, stroke, cerebral embolism, pulmonary embolism, ischemia and angina (including unstable angina).

The compounds of the invention are also useful as inhibitors of blood coagulation in an ex vivo situation such as, for example, the storage of whole blood or other biological samples suspected to contain Factor Xa and in which coagulation is detrimental.

WO 98/21188 describes a range of Factor Xa inhibitors. Further particular examples of this type of compound including 1-(5-chloroindol-2-ylsulphonyl)-4-[4-(6-oxo-1H-pyridazin-3-yl)benzoyl]piperazine are described in WO 99/57113. The applicants have found however, that by further derivatising the compounds of this type, enhanced properties may be obtained.

The present invention provides a compound of formula (I)

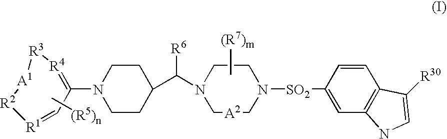

wherein R¹, R², R³ and R⁴ are independently selected from carbon and nitrogen, and where at least one of R¹, R², R³ and R⁴ is nitrogen;
A¹ is a single bond or a double bond;
n is 0, 1, 2 or 3;
each R⁵ is independently selected from hydrogen, halogen, C₁₋₃alkyl, oxo, oxy, oxido and thioxo;
R⁶ is hydrogen or oxo;
m is 0, 1, 2 or 3;
A² is a single bond or a double bond;
each R⁷ is independently selected from hydrogen, hydroxy, oxo, C₁₋₅alkyl, carboxy, cyano, tetrazolyl, N—C₁₋₅ alkyltetrazolyl, oxazolyl, C₁₋₅ oxazolyl, isoxazolyl, C₁₋₅ isoxazolyl, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅ alkyl)carbamoyl, carbamoylC₁₋₄alkyl,
C₁₋₅alkylcarbamoylC₁₋₄alkyl, di(C₁₋₅alkyl)carbamoylC₁₋₄alkyl, hydroxyC₁₋₅alkylcarbamoyl, C₁₋₅alkoxyC₁₋₅alkylcarbamoyl, hydroxyC₁₋₅alkylcarbamoylC₁₋₄alkyl, C₁₋₅alkoxyC₁₋₅alkylcarbamoylC₁₋₄alkyl, —CONR⁸(CH₂)ₓS(O)ₚR⁹, —CONH(CH₂)_{q}NR¹⁰R¹¹, —C₁₋₅alkyl-Y¹, —COOCHR¹⁷R¹⁸ and —CONR¹⁷R¹⁸:

wherein x represents an integer 0 to 4;

p is 0, 1 or 2;

q represents an integer 2 to 4;

R⁸ represents hydrogen or C₁₋₃ alkyl;

R⁹ represents C₁₋₅alkyl or phenyl; or

R⁸ and R⁹ may together form a C₁₋₅alkylene group;

R¹⁰ and R¹¹ independently represent hydrogen, C₁₋₅alkyl, phenyl, C₁₋₅alkylphenyl, S(O)ₚR⁹, COR¹² or a 5- or 6-membered monocyclic heteroaryl ring containing up to 3 heteroatoms selected from nitrogen, oxygen and sulphur;

R¹² represents hydrogen, C₁₋₅alkyl or phenyl;

Y¹ represents S(O)ₚR⁹, NHS(O)₂R⁹, NHCOR¹³, O(CH₂)ᵣR¹⁴, azetidino, pyrrolidin-1-yl, piperidino, morpholino, thiamorpholino, 1-oxothiamorpholino, 1,1-dioxothiamorpholino, piperazin-1-yl or C₁₋₅alkylamino,

R¹³ represents C₁₋₅alkyl, phenyl or C₁₋₅alkylphenyl;

r represents an integer 1 to 4;

when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;

wherein any phenyl group within R⁷ is independently substituted by 0, 1 or 2 substituents selected from halogeno, trifluoromethyl, cyano, C₁₋₅alkyl and C₁₋₅alkoxy;

R¹⁵ and R¹⁶ independently represent hydrogen or C₁₋₅alkyl;

R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached, a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅allyl, C₁₋₅alkoxyC₁₋₅allyl, carboxyC₁₋₅alkyl, C₁₋₅alkoxyoxoC₁₋₆alkyl, and carbamoylC₁₋₅alkyl;

R³⁰ is hydrogen, amino, methyl or halogen;
or a pharmaceutically acceptable salt thereof.
In this specification the term “alkyl” includes both straight and branched chain alkyl groups but references to individual alkyl groups such as “propyl” are specific for the straight chain version only. An analogous convention applies to other generic terms.
For the avoidance of doubt, the atoms of the indolyl ring appearing in formula (I) is numbered as drawn below:

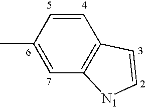

6-indolyl

It is to be understood that certain of the compounds of the formula (I) defined above can exist in solvated as well as unsolvated forms such as, for example, hydrated forms. It is to be understood that the invention encompasses all such solvated forms, which possess Factor Xa inhibitory activity.

It is further to be understood that, insofar as certain of the compounds of the formula (I) defined above may exist in optically active or racemic forms by virtue of one or more asymmetric carbon atoms, the invention encompasses any such optically active or racemic form which possesses Factor Xa inhibitory activity. The synthesis of optically active forms may be carried out by standard techniques of organic chemistry well known in the art, for example by synthesis from optically active starting materials or by resolution of a racemic form.

Further, “tautomer” or “tautomerism” refers to the coexistence of two (or more) compounds that differ from each other only in the position of one (or more) mobile atoms and in electron distribution, i.e. different tautomeric forms. An example may be keto-enol tautomers.
Moreover, it is also to be understood that, insofar as certain of the compounds of the formula (I) defined above may exist in various tautomeric forms, the invention encompasses any such tautomeric forms which possesses Factor Xa inhibitory activity.

Compounds of the invention are potent inhibitors of Factor Xa, and may have improved selectivity over oxido squalene cyclase, better solubility and/or less cytochrome P 450 (CYP₄₅₀) inhibition and/or Caco2-permeability than some related compounds. Caco2 is a cell line which mimics transport over the gut wall.

Suitable values in the compound of formula (I):

| | |
|---|---|
| for halogen: | fluoro, chloro, bromo, iodo; |
| for C₁₋₃alkyl (also | methyl, ethyl, propyl, isopropyl; |
| as in e.g. oxoC₁₋₃alkyl): | |
| for C₁₋₄alkyl (also | methyl, ethyl, propyl, isopropyl, n-butyl, |
| as in e.g. oxoC₁₋₄alkyl): | secbutyl, isobutyl, tertbutyl; |
| for C₁₋₅alkyl (also | C₁₋₄alkyl (as above), C₁₋₃alkyl (as above), |
| as in e.g. oxoC₁₋₅alkyl): | n-butyl, isobutyl, pentyl, 2-pentyl, 3- |
| | pentyl, 2-methyl-1-butyl, isopentyl, |
| | neopentyl, 3-methyl-2-butyl, 2-methyl-2- |
| | butyl; |
| for C₁₋₃alkoxy: | methoxy, ethoxy, propoxy, isopropoxy; |
| for C₁₋₄alkoxy: | C₁₋₃alkoxy (as above), n-butoxy, |
| | secbutoxy, isobutoxy, terbutoxy; |
| for C₁₋₅alkoxy: | C₁₋₄alkoxy (as above), C₁₋₃alkoxy (as |
| | above), pentoxy, 2-pentoxy, 3-pentoxy, 2- |
| | methyl-1-butoxy, isopentoxy, neopentoxy, |
| | 3-methyl-2-butoxy, 2-methyl-2-butoxy; |

for 4-, 5-, 6- or 7-membered heterocyclic ring: azetidine, pyrrolidine, morpholine, piperazine, azepane, [1,4]-diazepane, tetrahydro-pyran, or piperidin.
Moreover, the term “oxido” denotes a ⁻O-group (ion) and the term “carbamoyl” denotes a H₂N—C(O)-group.
In an embodiment of the invention a compound of formula (I) is disclosed where one or two of R¹, R², R³ and R⁴ is/are nitrogen.
A further embodiment of the invention discloses a compound of formula (I) wherein at least one of R¹, R² and R³ is nitrogen.
In a further embodiment of the invention a compound of formula (I) is disclosed wherein both R³ and R⁴ are nitrogen.
In still a further embodiment of the invention a compound of formula (I) is disclosed wherein R³ is nitrogen.
In even a further embodiment of the invention a compound of formula (I) is disclosed wherein both R¹ and R⁴ are nitrogen.
A further embodiment of the invention discloses a compound of formula (I) wherein R² is nitrogen.
In an embodiment of the invention a compound of formula (I) is disclosed wherein A¹ is a single bond.
In a further embodiment of the invention a compound of formula (I) is disclosed wherein A¹ is a double bond.
A further embodiment of the invention discloses a compound of formula (I) wherein n is 0, 1 or 2.
In a further embodiment of the invention a compound of formula (I) is disclosed where one of R⁵ is oxo.
In a even further embodiment of the invention said R⁵ being oxo is positioned at R².
In a further embodiment of the invention one of R⁵ is C₁₋₃alkyl, e.g. methyl, ethyl, or propyl.
In a even further embodiment of the invention one of R⁵ is halogen, e.g. fluoro, chloro or bromo.
In still a further embodiment of the invention one of R⁵ is oxido.
In a further embodiment of the invention a compound of formula (I) is disclosed where n is at least 2, one R⁵ is C₁₋₃alkyl, e.g. methyl, ethyl, or propyl, and the other R⁵ is oxo.
A further embodiment of the invention discloses a compound of formula (I) wherein m is 0, 1 or 2.
An even further embodiment of the invention discloses a compound of formula (I) wherein m is 2 or 3.
In a further embodiment of the invention a compound of formula (I) is disclosed where R⁶ is hydrogen and at least one of R⁷ is oxo.
In a further embodiment of the invention a compound of formula (I) is disclosed where m is 1, 2 or 3 and each R⁷ is independently selected from hydrogen, hydroxy, oxo, C₁₋₅alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷R¹⁸ and —CONR¹⁷R¹⁸:

wherein

R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 pr 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.
A further embodiment of the invention discloses a compound of formula (I) wherein one R⁷ is oxo, and at least one further R⁷ is selected from hydroxy, oxo, C₁₋₅alkyl, carboxy, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, carbamoylC₁₋₄alkyl, C₁₋₅alkylcarbamoylC₁₋₄alkyl, di(C₁₋₅alkyl)carbamoylC₁₋₄alkyl, hydroxyC₁₋₅alkylcarbamoyl, C₁₋₅alkoxyC₁₋₅alkylcarbamoyl, hydroxyC₁₋₅alkylcarbamoylC₁₋₄alkyl, C₁₋₅alkoxyC₁₋₅alkylcarbamoylC₁₋₄alkyl, —CONR⁸(CH₂)ₓS(O)ₚR⁹, —CONH(CH₂)_{q}NR¹⁰R¹¹, —C₁₋₅alkyl-Y¹, —COOCHR¹⁷R¹⁸ and —CONR¹⁷R¹⁸:

wherein x represents an integer 0 to 4;

p is 0, 1 or 2;

q represents an integer 2 to 4;

R⁸ represents hydrogen or C₁₋₃alkyl;

R⁹ represents C₁₋₅alkyl or phenyl; or

R⁸ and R⁹ may together form a C₁₋₅alkylene group;

R¹⁰ and R¹¹ independently represent hydrogen, C₁₋₅alkyl, phenyl, C₁₋₅alkylphenyl, S(O)ₚR⁹, COR¹² or a 5- or 6-membered monocyclic heteroaryl ring containing up to 3 heteroatoms selected from nitrogen, oxygen and sulphur;

R¹² represents hydrogen, C₁₋₅alkyl, phenyl or C₁₋₅alkylphenyl;

Y¹ represents S(O)ₚR⁹, NHS(O)₂R⁹, NHCOR¹³, O(CH₂)ᵣR⁴, pyrrolidin-1-yl, piperidino, morpholino, thiamorpholino, 1-oxothiamorpholino, 1,1-dioxothiamorpholino or piperazin-1-yl,

R¹³ represents C₁₋₅alkyl, phenyl or C₁₋₅alkylphenyl;

r represents an integer 1 to 4;

when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;

wherein any phenyl group within R⁷ is independently substituted by 0, 1 or 2 substituents selected from halogeno, trifluoromethyl, cyano, C₁₋₅alkyl and C₁₋₅alkoxy;

R¹⁵ and R¹⁶ independently represent hydrogen or C₁₋₅alkyl;

R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.
Still a further embodiment of the invention discloses a compound of formula (I) wherein one R⁷ is oxo, and at least one further R⁷ is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —CONR⁸(CH₂)ₓS(O)ₚR⁹, —CONH(CH₂)_{q}NR¹⁰R¹¹, —C₁₋₅alkyl-Y¹, —COOCHR¹⁷R¹⁸ and —CONR¹⁷R¹⁸:

wherein x represents an integer 0 to 4;

p is 0, 1 or 2;

q represents an integer 2 to 4;

R⁸ represents hydrogen or C₁₋₃alkyl;

R⁹ represents C₁₋₅alkyl or phenyl; or

R⁸ and R⁹ may together form a C₁₋₅alkylene group;

R¹⁰ and R¹¹ independently represent hydrogen, C₁₋₅ alkyl, phenyl, C₁₋₅alkylphenyl, S(O)ₚR⁹, COR¹² or a 5- or 6-membered monocyclic heteroaryl ring containing up to 3 heteroatoms selected from nitrogen, oxygen and sulphur;

R¹² represents hydrogen, C₁₋₅alkyl, phenyl or C₁₋₅alkylphenyl;

Y¹ represents S(O)ₚR⁹, NHS(O)₂R⁹, NHCOR¹³, O(CH₂)ᵣR¹⁴, pyrrolidin-1-yl, piperidino, morpholino, thiamorpholino, 1-oxothiamorpholino, 1,1-dioxothiamorpholino or piperazin-1-yl,

R¹³ represents C₁₋₅alkyl, phenyl or C₁₋₅alkylphenyl;

r represents an integer 1 to 4;

when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;

wherein any phenyl group within R⁷ is independently substituted by 0, 1 or 2 substituents selected from halogeno, trifluoromethyl, cyano, C₁₋₅alkyl and C₁₋₅alkoxy;

R¹⁵ and R¹⁶ independently represent hydrogen or C₁₋₅alkyl;

R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

A further embodiment of the invention discloses a compound of formula (I) wherein one R⁷ is oxo, and at least one further R⁷ is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷R¹⁸ and —CONR¹⁷R¹⁸R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.
An even further embodiment of the invention discloses a compound of formula (I) wherein one R⁷ is oxo, and at least one further R⁷ is selected from carboxy, hydroxyC₁₋₅alkyl, C₁₋₅alkoxyoxoC₁alkyl, carbamoyl, C₁₋₅alkylcarbamoyl, di(C₁₋₅ alkyl)carbamoyl, hydroxyC₁₋₅ alkylcarbamoyl and C₁₋₅ alkoxyC₁₋₅alkylcarbamoyl.
A still further embodiment of the invention discloses a compound of formula (I) wherein one R⁷ is oxo, and at least one further R⁷ is selected from —COOCHR¹⁷R¹⁸ and —CONR¹⁷R¹⁸:R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0 or 1 additional hetero oxygen, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅allyl, C₁₋₅alkoxyoxoC₁₋₆alkyl, and carbamoylC₁₋₅alkyl.
In still a further embodiment of the invention R⁶ is oxo.
A further embodiment of the invention discloses a compound of formula (I) wherein R⁶ is oxo and each R⁷ is independently selected from hydrogen, hydroxy, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, and C₁₋₅alkoxyC₁₋₅alkylcarbamoyl.
In a further embodiment of the invention R⁶ is oxo and one R⁷ is hydroxy.
In a even further embodiment of the invention m is 0.
In a further embodiment of the invention A² is a single bond.
A further embodiment of the invention discloses a compound of formula (I) wherein m is 0 and A² is a double bond.
Even a further embodiment of the invention discloses a compound of formula (I) wherein R³⁰ is halogen, e.g. fluoro, chloro or bromo.
Said heterocyclic ring formed from R¹⁷ and R¹⁵ is, for example, azetidine, pyrrolidine, morpholine, piperazine, azepane, [1,4]-diazepane, tetrahydro-pyran, or piperidin.
A further embodiment of the invention discloses a compound of formula (I) which is:
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester,
6-{4-[4-(3-Chloro-1H-indole-6-sulfonyl)-3-hydroxy-piperazine-1-carbonyl]-piperidin-1-yl}-2-methyl-2H-pyridazin-3-one,
6-{4-[4-(3-Chloro-1H-indole-6-sulfonyl)-2-hydroxy-piperazine-1-carbonyl]-piperidin-1-yl}-2-methyl-2H-pyridazin-3-one,
6-{4-[4-(3-Chloro-1H-indole-6-sulfonyl)-2-oxo-piperazin-1-ylmethyl]-piperidin-1-yl}-2-methyl-2H-pyridazin-3-one,
4-[4-(3-Chloro-1H-indole-6-sulfonyl)-piperazine-1-carbonyl]-5′-methyl-3,4,5,6-tetrahydro-2H,1′H-[1,3′]bipyridinyl-6′-one,
5-{4-[4-(3-Chloro-1H-indole-6-sulfonyl)-piperazine-1-carbonyl]-piperidin-1-yl}-3-methyl-1H-pyrazin-2-one,
6-{4-[4-(3-Chloro-1H-indole-6-sulfonyl)-piperazine-1-carbonyl]-piperidin-1-yl}-2-methyl-2H-pyridazin-3-one,
6-{4-[4-(1H-Indole-6-sulfonyl)-piperazine-1-carbonyl]-piperidin-1-yl}-2-methyl-2H-pyridazin-3-one,
6-{4-[4-(3-Chloro-1H-indole-6-sulfonyl)-piperazine-1-carbonyl]-piperidin-1-yl}-2H-pyridazin-3-one,
[4-(3-Chloro-1H-indole-6-sulfonyl)-piperazin-1-yl]-(1′-oxy-3,4,5,6-tetrahydro-2H-[1,4′]bipyridinyl-4-yl)-methanone,
[4-(3-Chloro-1H-indole-6-sulfonyl)-piperazin-1-yl]-(2′-methyl-3,4,5,6-tetrahydro-2H-[1,4′]bipyridinyl-4-yl)-methanone,
[4-(3-Chloro-1H-indole-6-sulfonyl)-piperazin-1-yl]-(3′-chloro-3,4,5,6-tetrahydro-2H-[1,4′]bipyridinyl-4-yl)-methanone,
[4-(3-Chloro-1H-indole-6-sulfonyl)-piperazin-1-yl]-(3,4,5,6-tetrahydro-2H-[1,4′]bipyridinyl-4-yl)-methanone,
[4-(1H-Indole-6-sulfonyl)-piperazin-1-yl]-(3,4,5,6-tetrahydro-2H-[1,4′]bipyridinyl-4-yl)-methanone,
6-{4-[4-(3-Chloro-1H-indole-6-sulfonyl)-3,4-dihydro-2H-pyrazine-1-carbonyl]piperidin-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide,
6-{4-[4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(S)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide, (S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid isopropylamide,
6-{4-[2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[4-(3-Chloro-1H-indole-6-sulfonyl)-2-hydroxymethyl-6-oxo-piperazin-1-ylmethyl]-piperidine-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid tert-butyl ester,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester, or
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropyl ester.

A heterocyclic derivative of formula I, or pharmaceutically acceptable salt thereof, may be prepared by any process known to be applicable to the preparation of related compounds, such as those described in WO 98/21188 and WO 99/57113. Such procedures are provided as a further feature of the invention and are illustrated by the following representative processes in which, unless otherwise stated any functional group, for example amino, aminoalkyl, carboxy, indolyl or hydroxy, is optionally protected by a protecting group which may be removed when necessary.

Necessary starting materials may be obtained by standard procedures of organic chemistry and by reference to the processes used in the Examples.

For instance, the present invention provides a process for preparing a compound of formula (I) or a pharmaceutically acceptable salt thereof, which comprises the reaction, conveniently in the presence of a suitable base, of an amine of formula (II), with or without a protection of the indole nitrogen,

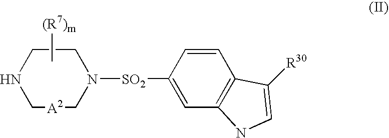

with a carboxylic acid of the formula (II),

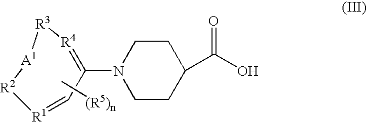

wherein the R-groups, A¹, A², n and m are as defined above in relation to formula (I), or a reactive derivative thereof.

A suitable reactive derivative of an acid of the formula (III) is, for example, an acyl halide, for example an acyl chloride formed by the reaction of the acid and an inorganic acid chloride, for example thionyl chloride; a mixed anhydride, for example an anhydride formed by the reaction of the acid with a chloroformate such as isobutyl chloroformate or with an activated amide such as 1,1′-carbonyldiimidazole; an active ester, for example an ester formed by the reaction of the acid and a phenol such as pentafluorophenol, an ester such as pentafluorophenyl trifluoroacetate or an alcohol such as N-hydroxybenzotriazole or N-hydroxysuccinimide; an acyl azide, for example an azide formed by the reaction of the acid and an azide such as diphenylphosphoryl azide; an acyl cyanide, for example a cyanide formed by the reaction of an acid and a cyanide such as diethylphosphoryl cyanide; or the product of the reaction of the acid and a carbodiimide such as N, N′ dicyclohexylcarbodiimide or N-(3 dimethylamino-propyl) N′ ethyl-carbodiimide.

The reaction is conveniently carried out in the presence of a suitable base such as, for example, an alkali or alkaline earth metal carbonate, also preferably carried out in a suitable inert solvent or diluent, for example methylene chloride or N,N-dimethylformamide, and at a temperature in the range, for example, −78° C. to 150° C., conveniently at or near ambient temperature.

Compounds of formula (IV)

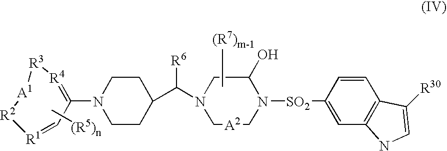

are suitably prepared by oxidative cleavage of the exocyclic double bond of formula (V), wherein the R-groups, A¹, A², n and m are as defined above in relation to formula (I). The in situ formed aldehyde spontaneously cyclize to form the more stable hemiaminal.

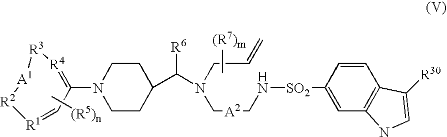

Typically, this reaction is carried out by reacting the compound of formula (V), wherein the possible positioning of (R⁷)ₘ corresponds to the possible positions of (R⁷)ₘ in the compound of formula (IV), to the with oxidizing agent such as sodium periodate/osmium tetroxide or ozone/dimethyl sulfide, also preferably carried out in a suitable inert solvent or diluent, for example tetrahydrofuran, methylene chloride, dioxane and at a temperature in the range, for example, −78° C. to 75° C., conveniently at or near ambient temperature.

Compounds of formula (VI), wherein R³⁰ is a halogen such as chloro or bromo,

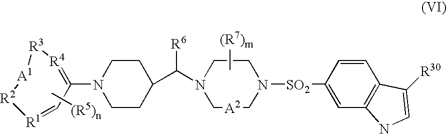

are prepared from compounds of formula (VII), wherein the R-groups, A¹, A², n and m are as defined above in relation to formula (I).

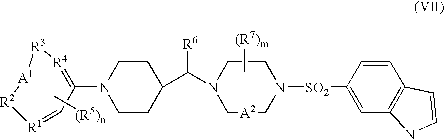

This reaction is conveniently carried out using the corresponding halogen succinimide in an inert solvent like dichloromethane or N,N-dimethylformamide at a temperature in the range −50° C.-100° C., conveniently at or near ambient temperature.

Compounds of formula (VIII)

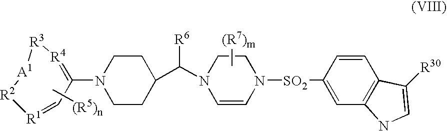

are prepared from compounds of formula (IV), wherein the R-groups, A¹, n and m are as defined above in relation to formula (I).

This reaction is carried out using acidic conditions conveniently in alcoholic solvents, typically methanol at a temperature in the range −50° C.-100° C., conveniently at or near ambient temperature.

In an alternative embodiment, amide derivatives from the exocyclic carboxylic acid of formula (IX), or a reactive derivative thereof,

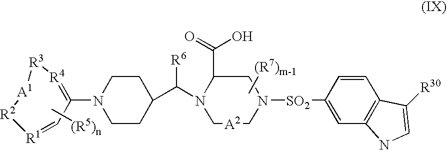

are prepared using conditions such as those described above for the conversion of II to III, wherein the R-groups, A¹, A², n and m are as defined above in relation to formula (I).

In an alternative embodiment, ester derivatives from the exocyclic carboxylic acid of formula (IX) or a reactive derivative thereof, wherein the R-groups, A¹, A², n and m are as defined above in relation to formula (I), are prepared using standard conditions following references found in Comprehensive Organic Transformations by Richard C. Larock. For example, for example treatment of (IX) in an readily available alcoholic solvent using acid catalysis, for example, using by saturation of the solvent by gaseous hydrogen chloride, furnish the corresponding ester derivatives. In case of hindered alcohols N,N-dimethylformamide dialkyl acetal is useful.

The preparation of derivatives of formula (I) are prepared by reaction a sulfonyl chloride derivative of formula (X), with our without a protecting group on the indolyl nitrogen,

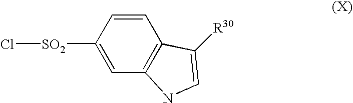

with an amine of formula (XI) or a salt thereof,

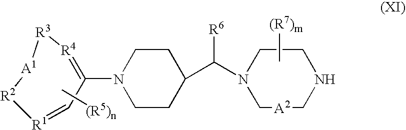

wherein the R-groups, A¹, A², n and m are as defined above in relation to formula (I).

This reaction is carried out using a base such as N,N-dimethyl aminopyridine, diisopropylethyl amine in inert solvents, typically dichloromethane and N,N-dimethylformamide at a temperature in the range −50° C.-100° C., conveniently at or near ambient temperature.

The preparation of derivatives of formula (XII), wherein the R-groups, A¹, n and m are as defined above in relation to formula (I),

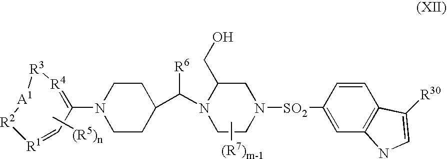

are prepared by reaction of a carboxylic acid derivative of formula (IX), or a reactive intermediate thereof e.g. a mixed anhydride formed by reacting (IX) with an alkyl chloroformate in situ, followed by addition of a reducing agent e.g. sodium borohydride.

This reaction is carried out in inert solvents, typically tetrahydrofuran at a temperature in the range −75° C.-50° C.

Compounds of formula (XIII),

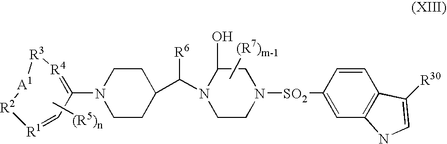

are suitably prepared by oxidative cleavage of the exocyclic double bond of formula (XIV), wherein the R-groups, A¹, n and m are as defined above in relation to formula (I) and the possible positioning of (R⁷)ₘ corresponds to the possible positions of (R⁷)ₘ in the compound of formula (IV). The in situ formed aldehyde spontaneously cyclize to form the more stable hemiaminal.

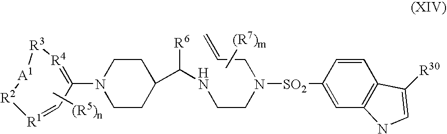

Typically, this reaction is carried out as described for the conversion of (V) to (IV).

When a pharmaceutically-acceptable salt of a compound of the formula (I) is required, it may be obtained, for example, by reaction of said compound with a suitable acid or base using a conventional procedure.

When an optically active form of a compound of the formula (I) is required, it may be obtained, for example, by carrying out one of the aforesaid procedures using an optically active starting material or by resolution of a racemic form of said compound using a conventional procedure, for example by the formation of diastereomeric salts, use of chromatographic techniques, conversion using stereospecific enzymatic processes, or by addition of temporary extra chiral group to aid separation.

The invention also relates to a process for preparing a compound of formula (I) which process comprises either

(a) reacting an amine of formula (II)

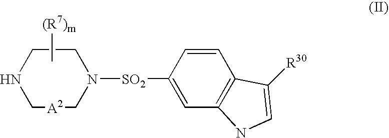

with an carboxylic acid of the formula (III)

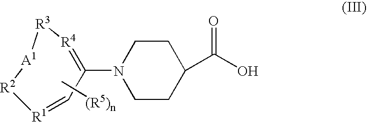

or a reactive derivative thereof; or
(b) reacting the compound of formula (V),

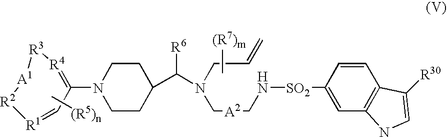

wherein the possible positioning of (R)ₘ corresponds to the possible positions of (R⁷)ₘ in the compound of formula (IV), with oxidazing agent such as sodium periodate/osmium tetroxide or ozone/dimethyl sulfide; or
(c) reacting the compound of formula (VII)

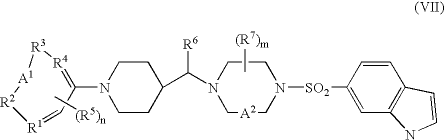

with the corresponding halogen succinimide; or
(d) carrying out a reaction with the compound of formula (IV)

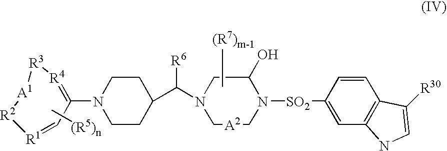

in acidic conditions; or
(e) where the compound of formula (I) is a compound of formula (IX),

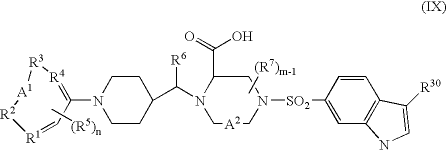

or a reactive derivative thereof,
conditions are used such as those described above in process (a), i.e. the conversion of II to III;
(f) where the compound of formula (I) is an ester derivative of the compound of formula (IX), the compound of formula (IX) are treated in an readily available alcoholic solvent using acid catalysis, for example, using by saturation of the solvent by gaseous hydrochloric acid, and using in the case of hindered alcohols N,N-dimethylformamide dialkyl acetal;
(g) reacting a sulfonyl chloride derivative of formula (X), with our without a protecting group on the indolyl nitrogen,

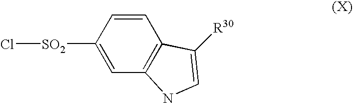

with an amine of formula (XI)

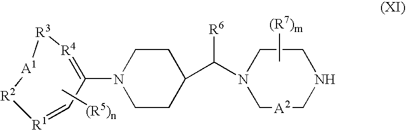

or a salt thereof;
(h) reacting a carboxylic acid derivative of formula (IX), or a reactive intermediate thereof e.g. a mixed anhydride formed by reacting (IX) with an alkyl chloroformate in situ, followed by addition of a reducing agent e.g. sodium borohydride; or
(i) oxidative cleaving of the exocyclic double bond of formula (XIV),

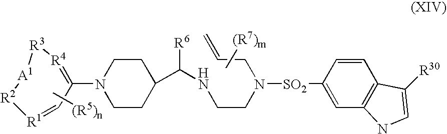

wherein the possible positioning of (R⁷)ₘ corresponds to the possible positions of (R⁷)ₘ in the compound of formula (IV).

As stated previously, the compounds of the formula (I) are inhibitors of the enzyme Factor Xa. The effects of this inhibition may be demonstrated using one or more of the standard procedures set out hereinafter:—

### a) Measurement of Factor Xa Inhibition

The FXa inhibitor potency was measured with a chromogenic substrate method, in a Plato 3300 robotic microplate processor (Rosys AG, CH-8634 Hombrechtikon, Switzerland), using 96-well, half-volume microtiter plates (Costar, Cambridge, Mass., USA; Cat No 3690). Stock solutions of test substance in DMSO (72 μL), 10 mmol/L, alternatively 1 mmol/L were diluted serially 1:3 (24+48 μL) with DMSO to obtain ten different concentrations, which were analyzed as samples in the assay, together with controls and blanks. As control sample melagatran was analysed. The dilutions of each test substance were analyzed consecutively, row-wise on the microtiter plate, with wash-cycles between substances to avoid cross-contamination. First 2 μL of test sample or DMSO for the blank were added, followed by 124 μL of assay buffer (0.05 mol/L Tris-hydrochloric acid pH 7.4 at 37° C., 5 mM CaCl₂, ionic strength 0.15 adjusted with NaCl, 0.1% bovine serum albumin, ICN Biomedicals, Inc, USA, 1 g/L) and 12 μL of chromogenic substrate solution (S-2765, Chromogenix, Mölndal, Sweden) and finally 12 μL of FXa solution (human FXa, Haematologic Technologies Inc., Essec Junction, Vt., USA), in buffer, was added, and the samples were mixed. The final assay concentrations were: test substance 0.0068-133, respectively 0.00068-13.3 μmol/L, S-2765 0.40 mmol/L (K_{M}=0.25 mmol/L) and FXa 0.1 μmol/L. The linear absorbance increase at 405 nm during 40 min incubation at 37° C. was used for calculation of percent inhibition for the test samples, as compared to references without inhibitor and/or enzyme. The IC₅₀-value, corresponding to the inhibitor concentration, which caused 50% inhibition of the FXa activity, was calculated by fitting the data to a three-parameter equation by Microsoft XLfit.

### b) Measurement of Thrombin Inhibition

The thrombin inhibitor potency was measured with a chromogenic substrate method developed in-house in principle as described in a) for FXa but using instead 0.3 mM of the chromogenic substrate solution S-2366 (Chromogenix, Mölndal, Sweden) and 0.1 nmol/L human thrombin (Haematologic Technologies Inc., Essec Junction, Vt., USA).

### c) Measurement of Anticoagulant Activity

An in vitro assay whereby human blood is collected and added directly to a sodium citrate solution (3.2 g/100 mL, 9 parts blood to 1 part citrate solution). Plasma is prepared by centrifugation (1000 g, 15 minutes) and stored at −80° C.) and an aliquot was rapidly thawed at 37° C. on the day of the experiment and kept on ice before addition to the coagulometer cups. Conventional prothrombin time (PT) tests are carried out in the presence of various concentrations of a test compound and the concentration of test compound required to double the clotting time is determined. Thromborel® S (Dade Behring, Liederbach, Germany) was reconstituted with 10 mL water. This solution was kept at 4° C. and was used within one week. Before the experiment the solution was kept at 37° C. for at least 30 minutes before start of the experiment. A ball coagulation timer KC 10A from Heinrich Amelung GmbH. (Lemgo, Germany) was used to study if the compounds could prevent coagulation in human plasma. The time for 50 μl plasma with compound to coagulate after addition of 100 μl Thromborel S, the Prothronibin Time or PTᵢ, is compared with the time it takes for pure plasma to coagulate, PT₀. With this technique the change in viscosity in the stirred solution is used to define clotting. The IC₅₀ is calculated from the curve of PTᵢ/PTₒ versus the inhibitor concentration in plasma, id est three times the final assay concentration.

### d) An In Vivo Measurement of Antithrombotic Activity

The abdoman is opened and the caval vein exposed. The thrombotic stimulus is partial stasis to the caval vein and a piece of filter paper soaked with ferric chloride and superimposed to the external surface of the vein. Thrombus size is determined as the thrombus wet weight at the end of the experiment. (Ref Thromb. Res. 2002; 107:163-168).
When tested in the above mentioned screen a) Measurement of Factor Xa Inhibition the compounds of the Examples gave IC₅₀ values for inhibition of Factor Xa activity of less than 10 μM, indicating that the compounds of the invention are expected to possess useful therapeutic properties.
Specimen results are shown in the following Table:

| Compound | IC₅₀ value (nM) |
|---|---|
| Example 3 | 4.8 |
| Example 6 | 98 |

A feature of the invention is a compound of formula (I), or a pharmaceutically acceptable salt thereof, for use in medical therapy.

According to a further feature of the invention there is provided a pharmaceutical composition which comprises a compound of formula (I), or a pharmaceutically acceptable salt thereof, in association with a pharmaceutically acceptable diluent or carrier.

The composition may be in a form suitable for oral use, for example a tablet, capsule, aqueous or oily solution, suspension or emulsion; for topical use, for example a cream, ointment, gel or aqueous or oily solution or suspension; for nasal use, for example a snuff, nasal spray or nasal drops; for vaginal or rectal use, for example a suppository; for administration by inhalation, for example as a finely divided powder such as a dry powder, a microcrystalline form or a liquid aerosol; for sub-lingual or buccal use, for example a tablet or capsule; or for parenteral use (including intravenous, subcutaneous, intramuscular, intravascular or infusion), for example a sterile aqueous or oily solution or suspension. In general the above compositions may be prepared in a conventional manner using conventional excipients.

The amount of active ingredient (that is a compound of the formula (I), or a pharmaceutically-acceptable salt thereof) that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient.

According to a further feature of the invention there is provided a compound of formula (I), or a pharmaceutically-acceptable salt thereof, for use in a method of treatment of the human or animal body by therapy.

The invention also includes the use of such an active ingredient (i.e. a compound of the formula a), or a pharmaceutically-acceptable salt thereof) in the production of a medicament for use in:—
(i) producing a Factor Xa inhibitory effect;
(ii) producing an anticoagulant effect;
(iii) producing an antithrombotic effect;
(iv) treating a Factor Xa mediated disease or medical condition;
(v) treating a thrombosis mediated disease or medical condition;
(vi) treating coagulation disorders; and/or

(vii) treating thrombosis or embolism involving Factor Xa mediated coagulation.

The invention also includes a method of producing an effect as defined hereinbefore or treating a disease or disorder as defined hereinbefore which comprises administering to a warm-blooded animal requiring such treatment an effective amount of an active ingredient as defined hereinbefore.

The size of the dose for therapeutic or prophylactic purposes of a compound of the formula (I) will naturally vary according to the nature and severity of the medical condition, the age and sex of the animal or patient being treated and the route of administration, according to well known principles of medicine. As mentioned above, compounds of the formula (I) are useful in the treatment or prevention of a variety of medical disorders where anticoagulant therapy is indicated. In using a compound of the formula (I) for such a purpose, it will generally be administered so that a daily oral dose in the range, for example, 0.5 to 100 mg/kg body weight/day is received, given if required in divided doses. In general lower doses will be administered when a parenteral route is employed, for example a dose for intravenous administration in the range, for example, 0.01 to 10 mg/kg body weight/day will generally be used. For preferred and especially preferred compounds of the invention, in general, lower doses will be employed, for example a daily dose in the range, for example, 0.1 to 10 mg/kg body weight/day. In general a preferred dose range for either oral or parenteral administration would be 0.01 to 10 mg/kg body weight/day.

Although the compounds of formula (I) are primarily of value as therapeutic or prophylactic agents for use in warm-blooded animals including man, they are also useful whenever it is required to produce an anticoagulant effect, for example during the ex vivo storage of whole blood or in the development of biological tests for compounds having anticoagulant properties.

The compounds of the invention may be administered as a sole therapy or they may be administered in conjunction with other pharmacologically active agents such as a thrombolytic agent, for example tissue plasminogen activator or derivatives thereof or streptokinase. The compounds of the invention may also be administered with, for example, a known platelet aggregation inhibitor (for example aspirin, a thromboxane antagonist or a thromboxane synthase inhibitor), a known hypolipidaemic agent or a known anti hypertensive agent.

The compounds of the invention may also be combined and/or co-administered with any antithrombotic agent(s) with a different mechanism of action, such as one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than FXa (e.g. synthetic thrombin, FVIIa, FXIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X1, P2Y1, P2Y12 [P2T]) antagonists; and inhibitors of carboxypeptidase U (CPU or TAIFa) and inhibitors of plasminogen activator inhibitor-1 (PAI-1).
The compounds of the invention may further be combined and/or co-administered with thrombolytics such as one or more of tissue plasminogen activator (natural, recombinant or modified), streptokinase, urokinase, prourokinase, anisoylated plasminogen streptokinase activator complex (APSAC), animal salivary gland plasminogen activators, and the like, in the treatment of thrombotic diseases, in particular myocardial infarction.
The invention further relates to a combination comprising a compound of formula (I) and any antithrombotic agent(s) with a different mechanism of action. Said antithrombotic agent(s) may be, for example, one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than FXa (e.g. synthetic thrombin, FVIIa, FXIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X1, P2Y1, P2Y12 [P2T]) antagonists; and inhibitors of carboxypeptidase U (CPU or TAFIa) and inhibitors of plasminogen activator inhibitor-1 (PAI-1).
Moreover, the invention further relates to a combination comprising a compound of formula (I) and thrombolytics, e.g. one or more of tissue plasminogen activator (natural, recombinant or modified), streptokinase, urokinase, prourokinase, anisoylated plasminogenstreptokinase activator complex (APSAC), animal salivary gland plasminogen activators.
Further, the invention also relates to a combination comprising a compound of formula (I) and thrombolytics, e.g. one or more of tissue plasminogen activator (natural, recombinant or modified), streptokinase, urokinase, prourokinase, anisoylated plasminogenstreptokinase activator complex (APSAC), animal salivary gland plasminogen activators, and the like, in the treatment of thrombotic diseases, in particular myocardial infarction.

The invention will now be illustrated in the following Examples in which, unless otherwise stated:—

(i) Yields are given for illustration only and are not necessarily the maximum attainable. Single node microwave irradiation was performed using either an Emrys Optimizer or a Smith Creator from Personal Chemistry. All solvents and reagents were used as purchased without purification unless noted;

(ii) The end-products have satisfactory high resolution mass spectral (HRMS) data as analysed on a Micromass QT of Micro spectrometer equipped with an Agilent 1100 LC system high performance liquid chromatography (HPLC). The spectrometer was continually calibrated with leucine enkephaline C₂₈H₃₇N₅O₇ (m/z 556.2771). MS conditions: Electrospray ionization, positive mode, capillary voltage 2.3 kV and desolvation temperature 150° C. Accurate mass was determined for positive ionization using leucine enkephaline (m/z 556.2771) as lock mass. Structures were confirmed by ¹H nuclear magnetic resonance (¹H NMR) spectra which were obtained with either a Varian Unity plus or a Varian Inova spectrometer operating at 400, 500 and 600 MHz respectively. Chemical shift values were measured on the delta scale; the following abbreviations have been used: s, singlet; d, doublet; t, triplet; q, quartet; sept, septet; m, multiplet;

(iii) Isolated intermediates were generally characterised as the end products with the exception of HRMS data;

(iv) Preparative reversed phase HPLC was performed using a Waters Prep LC 2000 with UV detection equipped with a 25 cm×2 cm or 30×5 cm C8 or C18 columns from Kromasil. Preparative chiral resolution using HPLC was performed using a Gilson 306 with UV detection equipped with either a Ciralpak AS (25×2 cm) (ester separations), a Chiralpak AD (25×2 cm) (amide separations) or a Chirobiotic R (25×2 cm) (carboxylic acid separation) column using 100% methanol or methanol/acetic acid/triethyl amine 100/0.1/0.05. All chiral separations were performed at 40° C.

### EXAMPLE 1

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid

The title product of Example 2, i.e. 4-(3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester, (35 mg, 0.061 mmol) was dissolved in tetrahydrofuran (0.75 mL) and a water solution of lithium hydroxide (1 M, 0.25 mL) was added. The mixture was stirred at room temperature for 1 hour. The reaction mixture was neutralized with acetic acid before purification with HPLC using a gradient of acetonitrile/5% acetonitrile water phase containing 0.1 M ammonium acetate, to give 30 mg (88%) of the title compound.

¹H NMR (500 MHz, dimethyl sulfoxide-d₆ as solvent and internal reference) δ (ppm) 0.88 (dq, 1H, J=4, 12 Hz), 1.02 (dq, 1H, J=4, 12 Hz), 1.23 (broad d, 1H, J=12 Hz), 1.44 (broad d, 1H, J=12 Hz), 1.52-1.62 (m, 1H), 2.34-2.54 (m, 3H), 2.98 (dd, 1H, J=4.4, 11.3 Hz), 3.35 (d, 1H, J=16.1 Hz), 3.57-3.70 (m, 5H), 3.77 (dd, 1H, J=3.8, 11.3 Hz), 6.75 (d, 1H, J=10.0 Hz), 7.38 (d, 1H, J=10.0 Hz), 7.46 (dd, 1H, J=1.6, 8.4 Hz), 7.70 (d, 1H, J=8.4 Hz), 7.85-7.87 (m, 2H).

HRMS (ESI+) calc. [M+H]⁺ 563.1474, found 563.1489.

### EXAMPLE 2

### (R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester

### A) (R)-4-(1-Benzenesulfonyl-3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester

To a mixture of (R)-1-[1-(1-methyl-6-oxo-1,6-dihydro-pyridazin-3-yl)-piperidin-4-ylmethyl]-6-oxo-piperazine-2-carboxylic acid methyl ester hydrochloride (185 mg, 0.46 mmol) in anhydrous dichloromethane/N,N-dimethylformamide 5:1 (4 mL) was added pyridine (0.10 mL, 1.2 mmol) at 0° C. under nitrogen atmosphere. To the mixture, a solution of 1-benzenesulfonyl-3-chloro-1H-indole-6-sulfonyl chloride (181 mg, 0.46 mmol) in anhydrous dichloromethane (2 mL) was added at 0° C., and the reaction mixture was stirred at room temperature for 20 minutes. The solvent was removed in vacuo before purification with HPLC using a gradient of acetonitrile/5% acetonitrile-water phase containing 0.1 M ammonium acetate, to give 150 mg (45%) of the sub-title compound after evaporation and freeze drying over night. The sub-title compound was used directly in step B.

### B)

The title compound was synthesized and purified essentially as in example 4, step E using the product from step A, i.e. (R)-4-(1-benzenesulfonyl-3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester, (150 mg, 0.21 mmol) as starting material to give 62 mg (51%).

¹H NMR (500 MHz, dimethyl sulfoxide-d₆ as solvent and internal reference) δ (ppm) 0.99 (dq, 1H, J=4, 12 Hz), 1.11 (dq, 1H, J=4, 12 Hz), 1.45 (broad d, 1H, J=12 Hz), 1.56 (broad d, 1H, J=12 Hz), 1.64-1.74 (m, 1H), 2.48-2.64 (m, 3H), 3.01 (dd, 1H, J=3.4, 12.2 Hz), 3.33-3.35 (m, 1H), 3.44 (s, 3H), 3.68 (s, 3H), 3.68-3.77 (m, 3H), 3.81 (d, 1H, J=16.1 Hz), 3.99 (d, 1H, J=12.2 Hz), 4.41 (t, 1H, J=2.7 Hz), 6.75 (d, 1H, J=10.0 Hz), 7.41 (d, 1H, J=10.0 Hz), 7.47 (dd, 1H, J=1.6, 8.4 Hz), 7.72 (d, 1H, J=8.4 Hz), 7.87 (d, 1H, J=1.2 Hz), 7.88 (s, 1H).

HRMS (ESI+) calc. [M+H]⁺ 577.1630, found 577.1622.

### EXAMPLE 3

### 6-{4-[4-(3-Chloro-1H-indole-6-sulfonyl)-3-hydroxy-piperazine-1-carbonyl]-piperidin-1-yl}-2-methyl-2H-pyridazin-3-one

### A) 1-(1-Methyl-6-oxo-1,6-dihydro-pyridazin-3-yl)-piperidine-4-carboxylic acid

Triethylamine (2.8 mL, 20 mmol) was added to a mixture of 6-chloro-2-methyl-2H-pyridazin-3-one (578 mg, 4.00 mmol) and piperidine-4-carboxylic acid (775 mg, 6.00 mmol) in 6.5 mL ethanol/water 3:1 in a microwave vial and heated at 180° C. for 15 hours. After cooling to room temperature 2 M sodium hydroxide (4 mL) was added to the reaction mixture. Ethanol and triethylamine were removed in vacuo and the basic aqueous solution was heated at 70° C. for 1.5 hours, diluted to 50 mL and washed twice with 20 mL ethyl acetate. The pH was adjusted to 5 using aqueous hydrochloric acid (a precipitate formed) and the volume of the mixture was reduced to 20 mL. The mixture was placed in the refrigerator over night and the solids were collected by filtration, washed with a small amount of water and dried under vacuum to give 497 mg of the sub-title compound (52%).

¹H NMR (400 Hz, dimethyl sulphoxide-d₆ as solvent and internal reference) δ (ppm) 1.54 (m, 2H), 1.84 (m, 2H), 2.40 (m, 1H), 2.76 (m, 2H), 3.47 (s, 3H), 3.74 (m, 2H), 6.78 (d, 1H, J=9.6 Hz), 7.47 (d, 1H, J=9.6 Hz).

### B) (2-{Allyl-[1-(1-methyl-6-oxo-1,6-dihydro-pyridazin-3-yl)-piperidine-4-carbonyl]-amino}-ethyl)-carbamic acid tert-butyl ester

1-Ethyl-3-(3-dimethylaminopropyl)carbodiimide hydrochloride (1.58 g, 8.22 mmol) was added to a stirred suspension of 1-(1-methyl-6-oxo-1,6-dihydro-pyridazin-3-yl)-piperidine-4-carboxylic acid (976 mg, 4.11 mmol), (2-allylamino-ethyl)-carbamic acid tert-butyl ester (1.18 g, 5.30 mmol) and 4-dimethylaminopyridine (2.01 g, 16.5 mmol) in anhydrous N,N-dimethylformamide (16 mL) at room temperature and the resulting suspension was stirred overnight. The resulting slightly cloudy solution was poured into a mixture of ice and water and the pH was adjusted to 4 using 1 M aqueous potassium hydrogensulfate while maintaining the temperature at 0° C. The aqueous solution was extracted with three portions of dichloromethane and the combined organic layers were washed with brine, dried, filtered, concentrated and pumped under high-vacuum to give the crude sub-title compound (1.93 g, 95% yield) as an oil which was used without further purification.

¹H NMR (500 MHz; chloroform-d as solvent and internal reference, major rotamer reported) δ (ppm) 7.10 (broad d, 1H, J=9.9 Hz), 6.83 (d, 1H, J=9.8 Hz), 5.72-5.86 (m, 1H), 5.24 (broad d, 1H, J=10.5 Hz), 5.10-5.18 (m, 1H), 4.95-5.01 (m, 1H), 3.96-4.00 (m, 2H), 3.81-3.87 (m, 2H), 3.63 (s, 3H), 3.41-3.50 (m, 2E, 3.21-3.30 (m, 2H), 2.70-2.84 (m, 2H), 2.53-2.61 (m, 1H), 1.84-1.96 (m, 2H), 1.69-1.82 (m, 2H), 1.43 (s, 9H)

### C) 1-(1-Methyl-6-oxo-1,6-dihydro-pyridazin-3-yl)-piperidine-4-carboxylic acid allyl-(2-amino-ethyl)-amide dihydrochloride

Crude (2-{allyl-[1-(1-methyl-6-oxo-1,6-dihydro-pyridazin-3-yl)-piperidine-4-carbonyl]-amino}-ethyl)-carbamic acid tert-butyl ester from step B (1.9 g, 3.9 mmol) was dissolved in 99.5% ethanol (20 mL) and cooled to 0° C. A 4 M solution of hydrochloric acid in dioxane (40 mL) was added dropwise and the reaction was stirred at 0° C. for S minutes and then for 1.5 hours at room temperature. The solvents were removed in vacuo and the residue was pumped under high-vacuum at 30° C. to give the crude sub-title compound (1.78 g, contains residual solvents, quantitative yield) as a foam which was used without further purification.

¹H NMR (500 MHz; methanol-d₄ as solvent and internal reference, major rotamer reported) δ (ppm) 7.69 (d, 1H, J=9.9 Hz), 7.10 (d, 1H, J=9.9 Hz), 5.90-5.98 (m, 1H), 5.29 (d, 1H, J=10.5 Hz), 5.22 (d, 1H, J=17.3), 4.90 (broad d, 1H, J=4.9 Hz), 4.06-4.12 (m, 2H), 3.77 (s, 3H), 3.60 (t, 2H, J=6.2 Hz), 3.10 (t, 2H, J=6.2 Hz), 2.85-2.98 (m, 3H), 1.73-1.86 (m, 4H).

### D) 1-(1-Methyl-6-oxo-1,6-dihydro-pyridazin-3-yl)-piperidine-4-carboxylic acid allyl-[2-(1-benzenesulfonyl-3-chloro-1H-indole-6-sulfonylamino)ethyl]-amide

A suspension of crude 1-(1-methyl-6-oxo-1,6-dihydro-pyridazin-3-yl)-piperidine-4-carboxylic acid allyl-(2-amino-ethyl)-amide dihydrochloride (224 mg, 0.457 mmol) from step C in anhydrous dichloromethane (3 mL) was added to a stirred solution of 1-benzenesulfonyl-3-chloro-1H-indole-6-sulfonyl chloride (140 mg, 0.370 mmol) and N,N-diisopropylethylamine (0.26 mL, 1.48 mmol) in anhydrous dichloromethane (1 mL) at 0° C. The reaction mixture was stirred at room temperature for 3 hours and then diluted with dichloromethane. Water was added and the aqueous layer was titrated to pH 4 using 1 M aqueous potassium hydrogensulfate and saturated aqueous sodium hydrogen carbonate. The layers were mixed thoroughly and then separated. The aqueous layer was extracted with a second portion of dichloromethane. The combined organic layers were washed with brine, dried, filtered and concentrated. The residue was purified by flash chromatography on silica gel eluted with 50:1 dichloromethane/methanol to give the sub-title compound (220 mg, 88.3%).

¹H NMR (500 MHz; chloroformed as solvent and internal reference, major rotamer reported) δ (ppm) 8.52 (d, 1H, J=1.4 Hz), 7.91-7.93 (m, 2H), 7.98 (dd, 1H, J=8.21, 1.5 Hz), 7.72 (s, 1H), 7.65 (d, 1H, J=8.4 Hz), 7.58-7.62 (m, 1H), 7.48-7.52 (m, 2H), 7.10 (d, 1H, J=9.9 Hz), 6.84 (d, 1H, J=9.9 Hz), 5.89 (t, 1H, 5.2 Hz), 5.77 ddt, 1H, J=17.2, 10.4, 4.8 Hz), 5.22 (broad d, 1H, J=10.2 Hz), 5.13 (broad d, 1H, J=17.2 Hz), 3.98-4.01 (m, 2H), 3.81-3.86 (m, 2H), 3.64 (s, 3H), 3.48 (t, 2H, J=5.8 Hz), 3.14 (q, 2H, J=5.6 Hz), 2.73 (td, 2H, J=12.7, 2.6 Hz), 2.55 (tt, 1H, J=11.3, 3.7 Hz), 1.68-1.92 (m, 4H).

### E) 1-(1-Methyl-6-oxo-1,6-dihydro-pyridazin-3-yl)-piperidine-4-carboxylic acid allyl-[2-(3-chloro-1H-indole-6-sulfonylamino)-ethyl]-amide

1-(1-Methyl-6-oxo-1,6-dihydro-pyridazin-3-yl)-piperidine-4-carboxylic acid allyl-[2-(1-benzenesulfonyl-3-chloro-1H-indole-6-sulfonylamino)-ethyl]-amide (220 mg, 0.33 mmol) from step D was treated essentially as in example 4, step E to give the sub-title compound (73 mg, 42% yield) as a solid.

¹H NMR (500 MHz; chloroform-d as solvent and internal reference, major rotamer reported) δ (ppm) 9.58 (broad s, 1H), 7.98 (broad s, 1H), 7.71 (d, 1H, J=8.5 Hz), 7.60 (dd, 1H, J=8.5, 1.4 Hz), 7.38-7.40 (m, 1H), 7.08 (d, 1H, J=9.9 Hz), 6.84 (d, 1H, J=9.9 Hz), 5.62-5.79 (m, 2H), 5.21 (d, 1H, J=10.2 Hz), 5.11 (d, 1H, J=17.2 Hz), 3.93-3.98 (m, 2H), 3.77-3.83 (m, 2H), 3.64 (s, 3H), 3.46 (t, 2H, J=5.7 Hz), 3.06-3.18 (m, 2H), 2.71 (td, 2H, J=12.8, 2.34 Hz), 2.53 (tt, 1H, J=11.3 Hz, 3.6 Hz), 1.65-1.92 (m, 4H).

### F)

1-(1-Methyl-6-oxo-1,6-dihydro-pyridazin-3-yl)-piperidine-4-carboxylic acid allyl-[2-(3-chloro-1H-indole-6-sulfonylamino)-ethyl]-amide (69 mg, 0.13 mmol) from step E was treated essentially as in example 4, step F to give the title compound (38 mg, 55% yield) as a solid.

¹H NMR (500 MHz; acetonitrile-d₃ as solvent and internal reference, two rotamers) δ (ppm) 9.99 (broad s, 1H), 7.99 (broad s, 1H), 7.67 (broad d, 1H, J=8.4 Hz), 7.57 (broad d, 1H, J=8.4 Hz), 7.52 (s, 1H), 7.20 (d, 1H, J=9.9 Hz), 6.69 (d, 1H, J=9.9 Hz), 5.49 (broad s, 1H), 4.33-4.45 (m, 1H), 3.93 (broad d, 1H, J=13.4 Hz), 3.72-3.80 (m, 2H), 3.49 (s, 3H), 3.42-3.53 (m, 1H), 3.29 (broad d, 0.6H, J=13.2 Hz, major rotamer), 3.17 (broad t, 0.4H, J=11.9 Hz, minor rotamer), 3.07 (broad t, 0.4H, J=11.9 Hz, minor rotamer), 2.95 (broad t, 0.6H, J=11.3 Hz, major rotamer), 2.85 (broad d, 0.4H, J=13.2 Hz), 2.68-2.80 (m, 4H), 1.55-1.80 (m, 4H).

HRMS (ESI+) calc. [M+H]⁺ 535.1525, found 535.1525.

### EXAMPLE 4

### 6-{4-[4-(3-Chloro-1H-indole-6-sulfonyl)-2-hydroxy-piperazine-1-carbonyl]-piperidin-1-yl}-2-methyl-2H-pyridazin-3-one

### A) (2-{[1-(1-Methyl-6-oxo-1,6-dihydro-pyridazin-3-yl)-piperidine-4-carbonyl]-amino}-ethyl)-carbamic acid tert-butyl ester

1-Ethyl-3-(3-dimethylaminopropyl)carbodiimide hydrochloride (695 mg, 3.62 mmol) was added to a stirred solution of 1-(1-methyl-6-oxo-1,6-dihydro-pyridazin-3-yl)-piperidine-4-carboxylic acid (430 mg, 1.81 mmol), N-boc-ethylenediamine (348 mg, 2.17 mmol) and 4-dimethylaminopyridine (886 mg, 3.62 mmol) in anhydrous N,N-dimethylformamide (8 mL) at room temperature and the solution was stirred overnight. The reaction mixture was poured onto ice-water and the pH was adjusted to pH 6 using 1 M aqueous potassium hydrogensulfate and the aqueous solution was extracted twice with ethyl acetate. The combined organic layers were washed with saturated aqueous sodium bicarbonate solution followed by brine, dried, filtered and concentrated to give crude (2-{[1-(1-methyl-6-oxo-1,6-dihydro-pyridazin-3-yl)-piperidine-4-carbonyl]-amino}-ethyl)-carbamic acid tert-butyl ester (400 mg). Further extraction of the aqueous reaction mixture with five portions of ethyl acetate essentially as described above gave an additional 180 mg of the crude sub-title compound to give a total of 580 mg (1.53 mmol, 84% yield) which was used without further purification.

¹H NMR (400 MHz; chloroform-d as solvent and internal reference) δ (ppm) 7.09 (d, 1H, J=10.1 Hz), 6.83 (d, 1H, J=10.1 Hz), 6.40-6.51 (m, 1H), 4.81-4.98 (m, 1H), 3.78-3.86 (m, 2H), 3.64 (s, 3H), 3.32-3.38 (m, 2H), 3.25-2.32 (m, 2H), 2.72-2.80 (m, 2H), 2.26 (tt, 1H, J=11.5, 3.8 Hz), 1.88-1.95 (m, 2H), 1.71-1.83 (m, 2H), 1.43 (s, 9H).

### B) 1-(1-Methyl-6-oxo-1,6-dihydro-pyridazin-3-yl)-piperidine-4-carboxylic acid (2-amino-ethyl)-amide hydrochloride

(2-{[1-(1-Methyl-6-oxo-1,6-dihydro-pyridazin-3-yl)-piperidine-4-carbonyl]-amino}-ethyl)-carbamic acid tert-butyl ester (580 mg, 1.52 mmol) from step A was suspended in 99.5% ethanol (5 mL) and cooled by an ice-bath. Hydrogen chloride (4 M solution in dioxane, 10 mL) was added dropwise and the reaction mixture was stirred at 0° C. for 30 minutes followed by 1 hour at room temperature. The solvents were removed in vacuo and the residue was dissolved in water and freeze-dried to give the crude sub-title compound (0.54 g, quantitative yield) as a solid which was used without further purification.

¹H NMR (400 MHz; dimethyl sulfoxide-d₆ as solvent and internal reference) δ (ppm) 8.19 (t, 1H, J=5.6 Hz), 8.11 (broad s, 3H), 7.51 (d, 1H, J=10.0 Hz), 6.81 (d, 1H, 10.0 Hz), 3.82-3.89 (m, 2H), 3.49 (s, 3H), 3.30 (q, 2H, J=6.1 Hz), 2.80-2.88 (m, 2H), 2.70 (dt, 2H, J=12.6, 2.4 Hz), 2.32 (tt, 1H, J=11.6, 3.9 Hz), 1.75-1.82 (m, 2H), 1.52-1.63 (m, 2H).

### C) 1-(1-Methyl-6-oxo-1,6-dihydro-pyridazin-3-yl)-piperidine-4-carboxylic acid [2-(1-benzenesulfonyl-3-chloro-1H-indole-6-sulfonylamino)-ethyl]-amide

A solution of 1-benzenesulfonyl-3-chloro-1H-indole-6-sulfonyl chloride (178 mg, 0.46 mmol) in anhydrous dichloromethane (2.5 mL) was added to a mixture of crude 1-(1-methyl-6-oxo-1,6-dihydro-pyridazin-3-yl)-piperidine-4-carboxylic acid (2-amino-ethyl)-amide hydrochloride (241 mg, 0.68 mmol) from step B and diisopropylethyl-amine (235 mg, 1.82 mmol) in anhydrous dichloromethane (1 mL) and the reaction was stirred at room temperature overnight. Dichloromethane and water was added and the aqueous layer was titrated to pH 4 using 1 M aqueous potassium hydrogensulfate and the layers were separated. The aqueous layer was extracted with two portions of dichloromethane and the combined organic layers were washed with brine, dried over magnesium sulfate, filtered and concentrated. The crude was purified by flash chromatography using a gradient of methanol in dichloromethane to give the sub-title compound (150 mg, 52% yield).

¹H NMR (400 MHz; chloroform-d as solvent and internal reference) δ (ppm) 8.54 (dd, 1H, J=1.6, 0.7 Hz), 7.92-7.95 (m, 2H), 7.79 (dd, 1H, J=8.3, 1.5 Hz), 7.74 (s, 1H), 7.69 (dd, 1H, J=8.3, 0.5 Hz), 7.60-7.65 (m, 1H), 7.50-7.55 (m, 2H), 7.11 (d, 1H, J=9.9 Hz), 6.85 (d, 1H, J=9.9 Hz), 6.11 (t, 1H, J=5.8 Hz), 5.36 (t, 1H, J=6.0 Hz), 3.81-3.87 (m, 2H), 3.65 (s, 3H), 3.48-3.43 (m, 2H), 3.08-3.13 (m, 2H), 2.72-2.80 (m, 2H), 2.27 (tt, 1H, J=11.6, 4.0 Hz), 1.86-1.94 (m, 2H), 1.71-1.83 (m, 2H).

### D) 1-(1-Methyl-6-oxo-1,6-dihydro-pyridazin-3-yl)-piperidine-4-carboxylic acid {2-[allyl-(1-benzenesulfonyl-3-chloro-1H-indole-6-sulfonyl)-amino]-ethyl}-amide

A mixture of crude 1-(1-methyl-6-oxo-1,6-dihydro-pyridazin-3-yl)-piperidine-4-carboxylic acid [2-(1-benzenesulfonyl-3-chloro-1H-indole-6-sulfonylamino)-ethyl]-amide (123 mg, 0.19 mmol) from previous step, allyl bromide (70 mg, 0.58 mmol) and potassium carbonate (115 mg, 0.835 mmol) in anhydrous acetonitrile was stirred overnight at room temperature. An additional 70 mg (0.58 mmol) of allyl bromide and 80 mg (0.58 mmol) of potassium carbonate were added in three portions and the reaction was again stirred overnight. The reaction mixture was diluted with dichloromethane and washed with water. The aqueous layer was extracted with dichloromethane and the combined organic layers were washed with brine, dried, filtered and concentrated to give the crude sub-title compound (0.14 g, quantitative yield) which was used without further purification.

¹H NMR (400 MHz; chloroform-d as solvent and internal reference) δ (ppm) 8.48 (dd, 1H, J=1.5, 0.8 Hz), 7.90-7.93 (m, 2H), 7.76 (s, 1H), 7.73 (AB dd, 1H, J=8.4, 1.4 Hz), 7.70 (AB dd, 1H, J=8.4, 0.6 Hz), 7.60 (m, 1H), 7.49-7.54 (m, 2H), 7.12 (d, 1H, J=9.9 Hz), 6.84 (d, 1H, J=9.9 Hz), 6.26 (broad t, 1H, J=5.1 Hz), 5.50-5.60 (m, 1H), 5.13-5.21 (m, 2H), 3.84-3.91 (m, 44, 3.65 (s, 3H), 3.41-3.46 (m, 2H), 3.23-3.28 (m, 2H), 2.76-2.84 (m, 2H), 2.34 (tt, 1H, J=11.7, 3.8 Hz), 1.96-2.03 (m, 2H), 1.78-1.90 (m, 2H).

### E) 1-(1-Methyl-6-oxo-1,6-dihydro-pyridazin-3-yl)-piperidine-4-carboxylic acid {2-[allyl-(3-chloro-1H-indole-6-sulfonyl)-amino]-ethyl}-amide

Crude 1-(1-methyl-6-oxo-1,6-dihydro-pyridazin-3-yl)-piperidine-4-carboxylic acid {2-[allyl-(1-benzenesulfonyl-3-chloro-1H-indole-6-sulfonyl)-amino]-ethyl}-amide (133 mg, 0.20 mmol) from step D was dissolved in anhydrous tetrahydrofuran and a 1 M solution of tetrabutylammonium fluoride (0.20 mL, 0.2 mmol) in tetrahydrofuran was added. The reaction was heated by single node microwave irradiation at 100° C. for 8 minutes. A second portion of 1 M tetrabutylammonium fluoride (0.025 mL, 0.025 mmol) in tetrahydrofuran was added and the reaction was heated for an additional 3 minutes at 100° C. The solvent was removed in vacuo and the crude was purified by preparative HPLC using a gradient of acetonitrile/5% acetonitrile-water phase containing 0.1 M ammonium acetate, to give the sub-title compound (65 mg, 62% yield) as a solid.

¹H NMR (400 MHz; chloroform-d as solvent and internal reference) δ (ppm) 8.67 (broad s, 1H), 7.91 (dd, 1H, J=1.5, 0.5 Hz), 7.76 (d, 1H, J=8.4 Hz), 7.57 (dd, 1H, J=8.46, 1.6 Hz), 7.42-7.43 (m, 1H), 7.12 (d, 1H, J=9.9 Hz), 6.85 (d, 1H, J=9.9 Hz), 6.35 (broad t, 1H, J=4.9 Hz), 5.59 (ddt, 1H, J=17.2, 10.2, 6.6 Hz), 5.12-5.20 (m, 2H), 3.84-3.90 (m, 4H), 3.66 (s, 3H), 3.39-3.44 (m, 2H), 3.26-3.29 (m, 2H), 2.76-2.84 (m, 2H), 2.33 (tt, 1H, J=11.7, 3.8 Hz), 1.94-2.01 (m, 2H), 1.77-1.88 (m, 2H).

### F)

A solution of sodium periodate (77 mg, 0.36 mmol) in water (0.5 mL) was added to a stirred solution of 1-(1-methyl-6-oxo-1,6-dihydro-pyridazin-3-yl)-piperidine-4-carboxylic acid {2-[allyl-(3-chloro-1H-indole-6-sulfonyl)-amino]-ethyl}-amide (60 mg, 0.11 mmol) from step E in tetrahydrofuran (1.5 mL). Osmium tetroxide (0.030 mL of a 2.5% wt solution in tert-butanol, 0.0030 mmol) was added and the reaction was stirred overnight during which a precipitate formed. The reaction mixture was diluted with dichloromethane and washed with water. The aqueous layer was extracted with ethyl acetate and the two organic layers were each washed with brine, combined, dried, filtered and concentrated. The crude was purified twice by preparative HPLC using a gradient of acetonitrile/5% acetonitrile-water phase containing 0.1 M ammonium acetate, to give the title compound (13.7 mg, 22.7% yield) as a solid.

¹H NMR (400 MHz; acetonitrile-d₃ as solvent and internal reference, two rotamers in 3:2 ratio) δ (ppm) 7.89 dd, 1H, J=1.4, 0.5 Hz), 7.70 (dd, 1H, J=8.4, 0.5 Hz), 7.54 (s, 1H), 7.45 (dd, 1H, J=8.4, 1.6 Hz), 7.30 (d, 1H, J=10.0 Hz), 6.79 (d, 1H, J=10.0 Hz), 5.87 (broad s, 0.4H, minor rotamer), 5.54 (broad s, 0.6H, major rotamer), 4.10-4.18 (broad d, 0.4H, 13.4 Hz), 3.66-3.86 (m, 4H), 3.51 (s, 3H), 3.46-3.61 (m, 1H), 3.10 (broad t, 1H, J=12.8 Hz), 2.57-2.79 (m, 3H), 2.20-2.51 (m, 2H), 1.40-1.75 (m, 4H).

HRMS (ESI+) calc. [M+H]⁺ 535.1525, found 535.1509.

### EXAMPLE 5

### 6-{4-[4-(3-Chloro-1H-indole-6-sulfonyl)-2-oxo-piperazin-1-ylmethyl]-piperidin-1-yl}-2-methyl-2H-pyridazin-3-one

### A) 1-(1-Methyl-6-oxo-1,6-dihydro-pyridazin-3-yl)-piperidine-4-carbaldehyde

To a solution of oxalyl chloride (5 mL 2.0 M solution in dichloromethane, 10 mmol) in anhydrous dichloromethane (35 mL) was added a solution of dimethyl sulfoxide (1.6 mL) in anhydrous dichloromethane (35 mL) at −78° C. dropwise under argon. During addition, the reaction temperature was kept below −65° C. The reaction mixture was stirred at −73° C. for 1 hour, whereupon a solution of 6-(4-hydroxymethyl-piperidin 1-yl)-2-methyl-2H-pyridazin-3-one (1.73 g, 7.74 mmol) in anhydrous dimethyl sulfoxide (20 mL) and anhydrous dichloromethane (20 mL) were added dropwise. The reaction mixture was stirred at between −70° C. and −65° C. for 1.5 hours then cooled to −73° C. and triethylamine (4.1 mL) was added dropwise. The reaction mixture was allowed to attain room temperature, water and dichloromethane were added. The organic phase was separated, and the aqueous phase was extracted twice with dichloromethane. The combined organic phases were washed with water, brine, dried and evaporated to dryness to give 1.7 (98%) of the sub-title compound.

¹H NMR (500 MHz, chloroform-d as solvent and internal reference) δ (ppm) 1.65 (dq, 2H, J=3.9, 13.8 Hz), 1.95 (dd, 2H, J=3.4, 13.5 Hz), 2.35-2.45 (m, 1H), 2.82-2.91 (m, 2H), 3.58 (s, 3H), 3.63-3.70 (m, 2H), 6.78 (d, 1H, J=9.9 Hz), 7.07 (d, 1H, J=9.9 Hz), 9.62 (s, 1H).

### B) (2-{[1-(1-Methyl-6-oxo-1,6-dihydro-pyridazin-3-yl)-piperidin-4-ylmethyl]-amino}-ethyl)-carbamic acid tert-butyl ester

To a solution of 1-(1-methyl-6-oxo-1,6-dihydro-pyridazin-3-yl)-piperidine-4-carbaldehyde (0.85 g, 3.4 mmol) from step A in anhydrous dichloromethane (16 mL) was added a solution of N-(tert-butoxycarbonyl)-1,2-diaminoethane (0.62 g, 3.8 mmol) in anhydrous dichloromethane (4 mL) and acetic acid (0.46 mL, 8.06 mmol) under argon. After stirring the resulting mixture at room temperature for 1 hour, sodium triacetoxy borohydride (2.85 g, 13.4 mmol) was added and the mixture was stirred over night. Water and dichloromethane were added, and then the aqueous phase was separated and freeze dried over night. The residue was suspended in dichloromethane, filtered and the solution was evaporated to dryness. The crude product was purified by column chromatography on silica gel using dichloromethane/methanol (100:10 and 100:15) as eluent to give 0.54 g (39%) of the sub-title compound.

¹H NMR (300 MHz, methanol-d₄ as solvent and internal reference) δ (ppm) 1.25-1.40 (m, 2H), 1.45 (s, 9H), 1.71-1.95 (m, 3H), 2.70-3.05 (m, 6H), 3.20-3.35 (m, 2H), 3.63 (s, 3H), 3.88-4.06 (m, 2H), 6.85 (d, 1H, J=9.90 Hz), 7.47 (d, 1H, J=9.90 Hz).

### C) (2-{(2-Chloro-acetyl)-[1-(1-methyl-6-oxo-1,6-dihydro-pyridazin-3-yl)-piperidin-4-ylmethyl]-amino}-ethyl)-carbamic acid tert-butyl ester

To a solution of (2-{[1-(1-methyl-6-oxo-1,6-dihydro-pyridazin-3-yl)-piperidin-4-ylmethyl]-amino}-ethyl)-carbamic acid tert-butyl ester (0.41 g, 1.12 mmol) from step B in anhydrous dichloromethane (12 mL) was added triethylamine (0.47 mL, 3.37 mmol) at 0° C. under argon. A solution of bromoacetyl chloride (0.27 g, 1.68 mmol) in anhydrous dichloromethane (2 mL) was added at 0° C. to the mixture dropwise, and then the reaction mixture was stirred at room temperature for 75 minutes. The reaction flask was cooled to 0° C., and water/dichloromethane was added. The organic phase was separated, washed with brine, dried and evaporated under reduced pressure. The crude product was purified by column chromatography on silica gel using dichloromethane/methanol (100:5) as eluent to give 0.22 g (44%) of the sub-title compound.

¹H NMR (300 MHz, methanol-d₄ as solvent and internal reference) δ (ppm) 1.25-1.40 (m, 2H), 1.42 (s, 9H), 1.60-2.05 (m, 3H), 2.65-2.85 (m, 2H), 3.17-3.38 (m, 4H), 3.42-3.54 (m, 2H), 3.62 (s, 3H), 3.85-4.10 (m, 3H), 3.28 (d, 1H, J=4.4 Hz), 6.85 (d, 1H, J=9.9 Hz), 7.45 (d, 1H, J=9.9 Hz).

### D) N-(2-Amino-ethyl)-2-chloro-N-[1-(1-methyl-6-oxo-1,6-dihydro-pyridazin-3-yl)-piperidin-4-ylmethyl]-acetamide hydrochloride

To a solution of (2-{(2-chloro-acetyl)-[1-(1-methyl-6-oxo-1,6-dihydro-pyridazin-3-yl)-piperidin-4-ylmethyl]-amino}-ethyl)-carbamic acid tert-butyl ester (0.22 g, 0.5 mmol) from step C in methanol (10 mL) was added a saturated methanolic hydrochloric acid (10 mL) at 0° C. After stirring at room temperature for 40 minutes, the solution was evaporated to dryness. The residue was dissolved in methanol and the solution evaporated to dryness to give 0.19 (98%) of the sub-title compound. The product was used directly in the next step.

### E) 2-Methyl-6-[4-(2-oxo-piperazin-1-ylmethyl)-piperidin-1-yl]-2H-pyridazin-3-one

To a solution of N-(2-amino-ethyl)-2-chloro-N-[1-(1-methyl-6-oxo-1,6-dihydro-pyridazin-3-yl)-piperidin-4-ylmethyl]-acetamide hydrochloride (0.19 g, 0.48 mmol) from step D in anhydrous N,N-dimethylformamide (3.5 mL) was added triethylamine (0.5 mL) at 0° C. under nitrogen. After stirring at room temperature for 2.5 hours, the solution evaporated to dryness and the crude product was purified by preparative HPLC using acetonitrile and ammonium acetate buffer (5:95 to 40:60) as eluent to give 90 mg (55%) of the sub-title compound.

¹H NMR (500 MHz, methanol-d₄ as solvent and internal reference) δ (ppm) 1.33 (dq, 2H, J=3.6, 12.3 Hz), 1.76 (d, 2H, J=12.7 Hz), 1.95-1.99 (m, 1H), 2.75-2.83 (m, 2H), 3.11 (t, 2H, J=5.2 Hz), 3.30-3.38 (m, 2H), 3.44 (t, 2H, J=5.2 Hz), 3.48 (s, 2H), 3.65 (s, 3H), 3.97 (d, 2H, J=13.0 Hz), 6.87 (d, 1H, J=9.9 Hz), 7.49 (d, 1H, J=10.1 Hz).

### F) 6-{4-[4-(1-Benzenesulfonyl-3-chloro-1H-indole-6-sulfonyl)-2-oxo-piperazin-1-ylmethyl]-piperidin-1-yl}-2-methyl-2H-pyridazin-3-one

To a solution of 2-methyl-6-[4-(2-oxo-piperazin-1-ylmethyl)-piperidin-1-yl]-2H-pyridazin-3-one (90 mg, 0.30 mmol) from step E in anhydrous N,N-dimethylformamide (2 mL) was added triethylamine (0.12 mL, 0.89 mmol) at 0° C. under nitrogen. To the mixture, a solution of 1-benzenesulfonyl-3-chloro-1H-indole-6-sulfonyl chloride (115 mg, 0.30 mmol) in anhydrous dichloromethane (2 mL) was added at 0° C., and the reaction mixture was stirred at room temperature for 1 hour. The reaction flask was cooled to 0° C., and water/dichloromethane was added. The organic phase was separated, washed with brine, dried and evaporated to dryness. The residue was suspended in ethanol, and the solids formed were filtered, washed with ethanol and dried in vacuo to give 130 mg (67%) of the sub-title compound.

### G)

A mixture of 6-{4-[4-(1-benzenesulfonyl-3-chloro-1H-indole-6-sulfonyl)-2-oxo-piperazin-1-ylmethyl]-piperidin-1-yl}-2-methyl-2H-pyridazin-3-one (0.13 g, 0.19 mmol) from step F and tetrabutylammonium fluoride (0.31 mL 1.0 M solution in tetrahydrofuran, 0.31 mmol) in tetrahydrofuran (2 mL) and ethanol (2 mL) was heated in a microwave oven at 100° C. for 12 minutes. The solution was concentrated in vacuo, and the residue was triturated with water to remove tetrabutylammonium fluoride. The crude product was purified by column chromatography on silica gel using dichloromethane/methanol (100:5) as eluent to give 72 mg (71%) of the title compound.

¹H NMR (500 MHz, methanol-d₄ as solvent and internal reference) δ (ppm) 1.10 (dq, 2H, J=3.8, 12.4 Hz), 1.39 (d, 2H, J=12.4 Hz), 1.65-1.74 (m, 1H), 2.55 (t, 2H, J=12.3 Hz), 3.19 (d, 2H, J=7.6 Hz), 3.35 (t, 2H, J=5.9 Hz), 3.44 (t, 2H, J=5.9 Hz), 3.61 (s, 3H), 3.74-3.80 (m, 4H), 6.82 (d, 1H, J=10.0 Hz), 7.38 (d, 1H, J=9.9 Hz), 7.56 (dd, 1H, J=1.5, 8.5 Hz), 7.59 (s, 1H), 7.74 (d, 1H, J=8.5 Hz), 7.95 (d, 1H, J=1.2 Hz).

HRMS (ESI+) calc. [M+H]⁺ 519.1576, found 519.1556.

### EXAMPLE 6

### 4-[4-(3-Chloro-1H-indole-6-sulfonyl)-piperazine-1-carbonyl]-5′-methyl-3,4,5,6-tetrahydro-2H,1′H-[1,3′]bipyridinyl-6′-one

### A) 5-bromo-2-methoxy-3-methyl-pyridine

A suspension of 2,5-dibromo-3-methylpyridine (2.08 g, 8.3 mmol) in a 2 M solution of sodium methoxide in methanol (17 mL) was heated by single node microwave irradiation at 120° C. for 40 minutes. The reaction mixture was poured onto a mixture of ice and 1 M aqueous hydrochloric acid and extracted with two portions of dichloro-methane. The combined organic layers were dried, filtered and concentrated in vacuo to give 1.57 g (89%) of the sub-title compound which was used without further purification.

¹H NMR (400 MHz; chloroform-d as solvent and internal reference) δ (ppm) 8.02 (d, 1H, J=2.3 Hz), 7.45-7.47 (m, 1H), 3.92 (s, 3H), 2.16 (broad s, 3H).

### B) 6′-Methoxy-5′-methyl-3,4,5,6-tetrahydro-2H-[1,3′]bipyridinyl-4-carboxylic acid ethyl ester

A stirred mixture of 5-bromo-2-methoxy-3-methyl-pyridine (525 mg, 2.47 mmol) from step A, ethyl isonipecotate (466 mg, 2.96 mmol), tris(dibenzylideneacetone)-palladium(0) (45 mg, 0.049 mmol), (S)-(−)-2,2-bis(diphenylphosphino)1,1-binaphtyl (62 mg, 0.099 mmol) and sodium tert-butoxide (333 mg, 3.46 mmol) was heated in anhydrous toluene (8 mL) at 70° C. under a nitrogen atmosphere for 2.5 hours. The reaction mixture was filtered through a short column of silica and the filtrate was concentrated in vacuo. The residue was purified by flash chromatography using a gradient of ethyl acetate in heptane to give the sub-title compound (235 mg, 34% yield).

¹H NMR (400 MHz; chloroform-d as solvent and internal reference) δ (ppm) 7.62 (dq, 1H, J=2.9, 0.5 Hz), 7.12 (dq, 1H, J=2.9, 0.7 Hz), 4.16 (q, 2H, J=7.1 Hz), 3.91 (s, 3H), 3.40-3.46 (m, 2H), 2.70 (ddd, 2H, J=12.1, 11.1, 2.8 Hz), 2.39 (tt, 1H, J=11.1, 8.2 Hz), 2.15-2.16 (m, 3H), 1.99-2.06 (m, 2H), 1.83-1.94 (m, 2H), 1.27 (t, 3H, J=7.1 Hz).

### C) 6′-Methoxy-5′-methyl-3,4,5,6-tetrahydro-2H-[1,3′]bipyridinyl-4-carboxylic acid hydrochloride

A solution of 6′-methoxy-5′-methyl-3,4,5,6-tetrahydro-2H-[1,3′]bipyridinyl-4-carboxylic acid ethyl ester (230 mg, 0.82 mmol) from step B and lithium hydroxide (59 mg, 2.5 mmol) in 67% aqueous tetrahydrofuran (6 mL) was stirred at room temperature for 3 hours. The reaction mixture was acidified to pH<2 by dropwise addition of 6 M hydrochloric acid. Most of the tetrahydrofuran was removed in vacuo and the remaining suspension containing the sub-title compound was freeze-dried and used without further purification.

¹H NMR (400 MHz; dimethyl sulfoxide-d₆ as solvent and internal reference) δ (ppm) 7.79 (broad s, 1H), 7.44 (broad s, 1H), 3.81 (s, 3H), 3.43-3.50 (m, 2H), 2.84-2.97 (m, 2H), 2.39-2.48 (m, 1 h), 2.11 (broad s, 3H), 1.92-2.00 (m, 2H), 1.69-1.81 (m, 2H).

### D) [4-(3-Chloro-1H-indole-6-sulfonyl)-piperazin-1-yl]-(6′-methoxy-5′-methyl-3,4,5,6-tetrahydro-2H-[1,3′]bipyridinyl-4-yl)-methanone

1-Ethyl-3-(3-dimethylaminopropyl)carbodiimide hydrochloride (348 mg, 1.8 mmol) was added to a stirred solution of 6′-methoxy-5′-methyl-3,4,5,6-tetrahydro-2H-[1,3′]bipyridinyl-4-carboxylic acid hydrochloride (crude from previous step, 0.83 mmol), 3-chloro-6-(piperazine-1-sulfonyl)-1H-indole (280 mg, 0.93 mmol) and 4-dimethylaminopyridine (504 mg, 4.1 mmol) in N,N-dimethylformamide (8 mL) at room temperature. After stirring for 3.5 hours the reaction mixture was filtered and purified by preparative HPLC using a gradient of acetonitrile/5% acetonitrile-water phase containing 0.1 M ammonium acetate to give the sub-title compound (262 mg, 57% yield).

¹H NMR (400 MHz; dimethyl sulfoxide-d₆ as solvent and internal reference) δ (ppm) 11.85 (broad s, 1H), 7.88 (s 1H), 7.84 (dd, 1H, J=1.6, 0.6 Hz), 7.72 (dd, 1H, J=8.4, 0.6 Hz), 7.53 (dd, 1H, J=2.9, 0.6 Hz), 7.44 (dd, 1H, J=8.4, 1.6 Hz), 7.24-7.26 (m, 1H), 3.77 (s, 3H), 3.51-3.64 (m, 4H), 3.43-3.49 (m, 2H), 2.83-2.93 (m, 4H), 2.52-2.67 (m, 3H), 2.07-2.08 (m, 3H), 1.53-1.61 (m, 4H).

### E)

A mixture of [4-(3-Chloro-1H-indole-6-sulfonyl)-piperazin-1-yl]-(6′-methoxy-5′-methyl-3,4,5,6-tetrahydro-2H-[1,3′]bipyridinyl-4-yl)-methanone (144 mg, 0.27 mmol) from step D and pyridine hydrochloride (375 mg, 3.24 mmol) was heated at 140° C. for 9.5 minutes in a preheated oil bath and the reaction mixture was cooled to room temperature. Water and dichloromethane was added together with a small amount of acetonitrile. The layers were separated and the aqueous layer was extracted with dichloromethane. The combined organic layers were washed with water, dried, filtered and concentrated in vacuo. The crude was purified by preparative HPLC using a gradient of acetonitrile/5% acetonitrile-water phase containing 0.1 M ammonium acetate to give 101 mg (68.4% yield) of the title compound as a solid after freeze-drying.

¹H NMR (400 MHz; dimethyl sulfoxide-d₆ as solvent and internal reference) δ (ppm) 11.13 (broad s, 2H), 7.87 (s 1H), 7.83 (dd, 1H, J=1.6, 0.6 Hz), 7.72 (dd, 1H, J=8.4, 0.6 Hz), 7.43 (dd, 1H, J=8.4, 1.6 Hz), 7.26-7.28 (m, 1H), 6.51 (d, 1H, J=3.1 Hz), 3.50-3.61 (m, 4H), 3.15-3.22 (m, 2H), 2.83-2.92 (m, 4H), 2.52-2.60 (m, 1H), 2.31-2.40 (m, 2H), 1.92-1.93 (m, 3H), 1.48-1.59 (m, 4H).

HRMS (ESI+) calc. [M+H]⁺ 518.1623, found 518.1625.

### EXAMPLE 7

### 5-{4-[4-(3-Chloro-1H-indole-6-sulfonyl)piperazine-1-carbonyl]-piperidin-1-yl}-3-methyl-1H-pyrazin-2-one

### A) 1-(5-Chloro-6-methyl-pyrazin-2-yl)-piperidine-4-carboxylic acid

2,5-Dichloro-3-methyl-pyrazine has been previously described by Sato et. al J. Het. Chem. 1986, 871. A mixture of 2,5-dichloro-3-methyl-pyrazine (880 mg, 5.40 mmol), ethyl isonipecotate (848 mg, 5.40 mmol) and triethylamine (1.64 g, 16.2 mmol) in 25% aqueous ethanol (15 mL) was heated using single node microwave irradiation at 170° C. for 40 minutes. The solvents were concentrated in vacuo to a volume of between 3.5 mL tetrahydrofuran was added until an almost clear solution was obtained. Solid lithium hydroxide (400 mg, 16.7 mmol) was added. The resulting suspension was stirred overnight at room temperature and the solution was acidified to pH 1. The solid material was filtered off, washed with water and dried under high-vacuum to give the crude sub-title compound (734 mg, 47% yield) as a solid which was used without further purification.

¹H NMR (400 MHz; dimethyl sulfoxide-d₆ as solvent and internal reference) δ (ppm) 12.23 (broad s, 1H), 7.94 (s, 1H), 4.12-4.22 (m, 2H), 2.95-3.05 (m, 2H), 2.41-2.63 (m, 1H), 2.39 (s, 3H), 1.83-1.94 (m, 2H), 1.46-1.59 (m, 2H).

### B) 1-(6-Methyl-5-oxo-4,5-dihydro-pyrazin-2-yl)-piperidine-4-carboxylic acid

A solution of crude 1-(5-chloro-6-methyl-pyrazin-2-yl)-piperidine-4-carboxylic acid from previous step (402 mg, 1.57 mmol) and potassium acetate (3.0 g, 31 mmol) in trifluoroacetic acid (15 mL) was heated using single node microwave irradiation at 120° C. for 10 hours. After cooling the solvent was removed in vacuo. The crude was purified by preparative HPLC using first 3% acetonitrile-water phase containing 0.1 M ammonium acetate and then a gradient of acetonitrile/5% acetonitrile-water phase containing 0.1 M ammonium acetate to give the sub-title compound (88 mg, 19% yield, 80% purity) which was used without father purification.

¹H NMR (400 MHz; dimethyl sulfoxide-d₆ as solvent and internal reference) δ (ppm) 6.83 (s, 1H), 3.61-3.69 (m, 2H), 2.51-2.59 (m, 2H), 2.22 (s, 3H), 2.11-2.24 (m, 1H), 1.77-1.85 (m, 2H), 1.46-1.58 (m, 2H)

### C)

1-(6-Methyl-5-oxo-4,5-dihydro-pyrazin-2-yl)-piperidine-4-carboxylic acid from step B was treated essentially as in example 6 step D to give the title compound (30 mg, 17% yield).

¹H NMR (400 MHz; dimethyl sulfoxide-d₆ as solvent and internal reference) δ (ppm) 7.88 (s, 1H), 7.83 (d, 1H, J=1.5 Hz), 7.72 (d, J=8.4 Hz), 7.44 (dd, J=8.4, 1.5 Hz), 7.00 (broad s, 1H), 3.77-3.89 (m, 2H), 3.51-3.63 (m, 4H), 2.84-2.94 (m, 4H), 2.62-2.72 (m, 1H), 2.51-2.59 (m, 2H), 2.22 (s, 3H), 1.42-1.60 (m, 4H).

HRMS (ESI+) calc. [M+H]⁺ 519.1576, found 519.1597.

### EXAMPLE 8

### 6-{4-[4-(3-Chloro-1H-indole-6-sulfonyl)-piperazine-1-carbonyl]-piperidin-1-yl}-2-methyl-2H-pyridazin-3-one and 6-{4-[4-(1H-Indole-6-sulfonyl)-piperazine-1-carbonyl]-piperidin-1-yl}-2-methyl-2H-pyridazin-3-one (the sub-title product of step D)

### A) 4-(4-Methyl-3-nitro-benzenesulfonyl)-piperazine-1-carboxylic acid tert-butyl ester

To a solution of tert-butyl 1-piperazinecarboxylate (7.11 g, 38.2 mmol) in anhydrous dichloromethane (100 mL) was added diisopropylethylamine (9.88 g, 76.4 mmol) under nitrogen. 4-methyl-3-nitrobenzenesulfonyl chloride (9.0 g, 38 mmol) in anhydrous dichloromethane (100 mL) was added to the solution at 0° C. drop wise. The reaction mixture was stirred at room temperature over night. After addition of water, the organic phase was separated, washed with water, brine, dried and evaporated under reduced pressure to give 13.8 g (94%) of the sub-title product.

¹H NMR (500 MHz, chloroform-d as solvent and internal reference): 1.42 (s, 9H), 2.70 (s, 3H), 3.0-3.07 (m, 4H), 3.50-3.57 (m, 4H), 7.56 (d, 1H, J=8.05 Hz), 7.86 (d, 1H, J=8.05 Hz), 8.32 (s, 1H).

### B) 4-(1H-Indole-6-sulfonyl)-piperazine-1-carboxylic acid tert-butyl ester

A solution of 4-(4-methyl-3-nitro-benzenesulfonyl)-piperazine-1-carboxylic acid tert-butyl ester (6.00 g, 15.6 mmol) from step A in N,N-dimethylformamide dimethyl acetal (40 mL) containing N,N-dimethylformamide (6 mL) was heated to 100° C. for 9 hours, then evaporated to dryness. The residue was then dissolved in tetrahydrofuran (65 mL) and methanol (65 mL), and then Raney nickel (3 spoonfuls) was added. Hydrazine monohydrate (10 mL) was added dropwise, keeping the internal temperature at 45° C. The reaction mixture was stirred at 45° C. for another 2.5 hours. After addition of tetrahydrofuran and methanol, the catalyst was filtered over Celite, and the solution evaporated to dryness. The residue was suspended in ethanol, and the solids filtered to give 4.69 g (82%) of the sub-title product.

¹H NMR (500 MHz, chloroform-d as solvent and internal reference): 1.40 (s, 9H), 2.94-3.04 (m, 4H), 3.46-3.55 (m, 4H), 6.67 (s, 1H), 7.46 (s, 1H), 7.47 (d, 1H, J=8.0 Hz), 7.77 (d, 1H, J=8.0 Hz), 7.91 (s, 1H), 8.83 (s, 1H).

### C) 6-(piperazine-1-sulfonyl)-1H-indole hydrochloride

To a mixture of 4-(1H-indole-6-sulfonyl)-piperazine-1-carboxylic acid tert-butyl ester (4.69 g, 12.8 mmol) from step B in ethanol (40 mL) was added saturated ethanol hydrogen chloride (100 mL) at 0° C. dropwise. After stirring for 105 minutes at room temperature, the solution was evaporated to dryness. The residue was suspended in ether and the solids formed were filtered to give 3.8 g (98%) of the sub-title product.

¹H NMR (300 MHz, methanol-d₄ as solvent and internal reference) δ (ppm) 3.20-3.37 (m, 8H), 6.62 (d, 1H, J=3.0 Hz), 7.43 (dd, 1H, J=1.5, 8.4 Hz), 7.56 (d, 1H, J=3.2 Hz), 7.79 (d, 1H, J=8.4 Hz), 7.92 (s, 1H).

### D) 6-{4-[4-(1H-Indole-6-sulfonyl)-piperazine-1-carbonyl]-piperidin-1-yl}-2-methyl-2H-pyridazin-3-one

To a mixture of 1-(1-methyl-6-oxo-1,6-dihydro-pyridazin-3-yl)-piperidine-4-carboxylic acid hydrochloride (80 mg, 0.29 mmol), and 6-(piperazine-1-sulfonyl)-1H-indole hydrochloride (97 mg, 0.32 mmol) from step C in anhydrous N,N-dimethylformamide (2 mL) was added diisopropylethylamine (150 mg, 1.17 mmol) and 2-(1H-benzotriazole-1-yl)-1,1,3,3-tetramethyluronium tetrafluoroborate (117 mg, 0.37 mmol) under argon. After stirring at room temperature for 30 minutes, the reaction flask was cooled to 0° C. and the reaction mixture was quenched by adding water. The solution was evaporated to dryness, and the crude product was purified by preparative HPLC using acetonitrile and ammonium acetate buffer (20:80 to 55:45) as eluent to give 140 mg (99%) of the sub-title product.

¹H NMR (500 MHz, methanol-d₄ as solvent and internal reference) δ (ppm) 1.56-1.67 (m, 4H), 2.71-2.83 (m, 3H), 2.94-3.05 (m, 4H), 3.59 (s, 3H), 3.61-3.71 (m, 4H), 3.89 (d, 2H, J=12.98 Hz), 6.59 (d, 1H, J=2.33 Hz), 6.81 (d, 1H, J=9.86 Hz), 7.36-7.43 (m, 3H), 7.52 (d, 1H, J=3.12 Hz), 7.74 (d, 1H, J=8.30 Hz), 7.87 (s, 1H).

HRMS (ESI+) calc. [M+H]⁺ 485.1965, found 485.1907.

### E) 6-{4-[4-(3-Chloro-1H-indole-6-sulfonyl)-piperazine-1-carbonyl]-piperidin-1-yl}-2-methyl-2H-pyridazin-3-one

To a solution of 6-{4-[4-(1H-indole-6-sulfonyl)-piperazine-1-carbonyl]-piperidin-1-yl}-2-methyl-2H-pyridazin-3-one (138 mg, 0.29 mmol) from step D in anhydrous N,N-dimethylformamide (2 mL) was added N-chlorosuccinimide (60 mg, 0.45 mmol) under argon. After stirring the reaction mixture for 4 hours and 15 minutes, the reaction flask was cooled to 0° C. and the reaction mixture was quenched by adding water. The solids formed were filtered, washed with water and purified by preparative HPLC using acetonitrile and ammonium acetate buffer (25:75 to 60:40) as eluent to give 80 mg (54%) of the title product.

¹H NMR (500 MHz, methanol-d₄ as solvent and internal reference) δ (ppm) 1.61-1.68 (m, 4H), 2.73-2.84 (m, 3H), 2.96-3.07 (m, 4H), 3.60 (s, 3H), 3.63-3.72 (m, 4H), 3.90 (d, 2H, J=13.0 Hz), 6.82 (d, 1H, J=9.9 Hz), 7.42 (d, 1H, J=10.1 Hz), 7.50 (d, 1H, J=8.6 Hz), 7.57 (s, 1H), 7.72 (d, 1H, J=8.3 Hz), 7.89 (s, 1H).

HRMS (ESI+) calc. [M+H]⁺ 519.1576, found 519.1610.

### EXAMPLE 9

### 6-{4-[4-(3-Chloro-1H-indole-6-sulfonyl)piperazine-1-carbonyl]-piperidin-1-yl}-2H-pyridazin-3-one

### A) 3-Chloro-6-(piperazine-1-sulfonyl)-1H-indole

To a solution of 6-piperazine-1-sulfonyl)-1H-indole hydrochloride (3.0 g, 9.94 mmol) in anhydrous N,N-dimethylformamide (9 mL) was added N-chlorosuccinimide (1.35 g, 10.1 mmol) under nitrogen. After stirring at room temperature for 40 minutes, the reaction flask was cooled to 0° C. and the reaction mixture was quenched by adding water. The solution was made alkaline by adding sodium bicarbonate, and solid material precipitated. The solids was filtered, washed with water, ethanol, ether and dried in vacuo to give 2.5 g (84%) of 3-chloro-6-piperazine-1-sulfonyl)-1H-indole.

¹H NMR (300 MHz, methanol-d₄ as solvent and internal reference) δ (ppm) 3.20-3.30 (m, 8H), 7.54 (dd, 1H, J=1.7, 8.6 Hz), 7.60 (s, 1H), 7.77 (dd, 1H, J=0.5, 8.4 Hz), 7.94 (d, 1H, J=0.5 Hz).

The hydrochloride salt was optionally prepared by adding 1 M hydrochloric acid to the neutral form dissolved in methanol followed by removal of solvents in vacuo.

### B)

To a mixture of 1-(6-oxo-1,6-dihydro-pyridazin-3-yl)-piperidine-4-carboxylic acid (60 mg, 0.27 mmol), and 3-chloro-6-piperazine-1-sulfonyl)-1H-indole hydrochloride (90 mg, 0.27 mmol) from step A in anhydrous NAN-dimethylformamide (2 mL) was added diisopropylethylamine (87 mg, 0.67 mmol) and 2-(1H-benzotriazole-1-yl)-1,1,3,3-tetramethyluronium tetrafluoroborate (117 mg, 0.37 mmol). After stirring at room temperature for 50 minutes, the reaction flask was cooled to 0° C. and the reaction mixture was quenched by adding water. The solids formed were filtered, washed with water and purified by column chromatography on silica gel using dichloromethane/methanol (100:4 and 100:7) as eluent to give 70 mg (52%) of the title product.

¹H NMR (500 MHz, methanol-d₄ as solvent and internal reference) δ (ppm) 1.59-1.71 (m, 4H), 2.72-2.84 (m, 3H), 2.96-3.08 (m, 4H), 3.61-3.73 (m, 4H), 3.82-3.93 (m, 2H), 6.80-6.87 (m, 1H), 7.44-7.54 (m, 2H), 7.57 (d, 1H, J=7.0 Hz), 7.70-7.76 (m, 1H), 7.89 (d, 1H, J=5.4 Hz).

HRMS (ESI+) calc. [M+H]⁺ 505.1419, found 505.1440.

### EXAMPLE 10

### [4-(3-Chloro-1H-indole-6-sulfonyl)-piperazin-1-yl]-(1′-oxy-3,4,5,6-tetrahydro-2H-[1,4′]bipyridinyl-4-yl)-methanone

A mixture of 1′-oxy-3,4,5,6-tetrahydro-2H-[1,4′]bipyridinyl-4-carboxylic acid (160 mg, 0.534 mmol) and 3-chloro-6-(piperazine-1-sulfonyl)-1H-indole (108 mg, 0.486 mmol) in dry N,N-dimethylformamide (3 mL) and dry tetrahydrofuran (1 mL) and was treated at room temperature with N,N-diisopropylethylamine (170 μL, 0.972 mmol) and O-(benzotriazol 1-yl)-N,N,N′N′-tetramethyluronium tetrafluoroborate (195 mg, 0.608 mmol). After 2 hours the reaction mixture was concentrated in vacuo. Crushed ice was added to the residue and the formed solid material was filtered, washed with water, dried, and subjected to reversed phase preparative HPLC (20→50% acetonitrile in 0.1 M aqueous ammonium acetate) to give 110 mg (45%) of the title product.

¹H NMR (600 MHz, methanol-d₄ as solvent and internal reference) δ (ppm) 1.59-1.72 (m, 4H), 2.88-2.93 (m, 1H), 2.98-3.04 (m, 6H), 3.63-3.71 (m, 4H), 3.92-3.96 (m, 2H), 6.91 (d, 2H, J=7.8 Hz), 7.49 (dd, 1H, J=1.4, 8.5 Hz), 7.56 (5, 1H), 7.72 (d, 1H, J=8.5 Hz), 7.88 (s, 1H), 7.92 (d, 2H, J=7.8 Hz).

HRMS (ESI+) calc. [M+H]⁺ 504.1467, found 504.1449.

### EXAMPLE 11

### [4-(3-Chloro-1H-indole-6-sulfonyl)-piperazin-1-yl]-(2′-methyl-3,4,5,6-tetrahydro-2H-[1,4′]bipyridinyl-4-yl)-methanone

A mixture of 2′-methyl-3,4,5,6-tetrahydro-2H-[1,4′]bipyridinyl-4-carboxylic acid (186 mg, 0.621 mmol) and 3-chloro-6-(piperazine-1-sulfonyl)-1H-indole (130 mg, 0.590 mmol) in dry N,N-dimethylformamide (3 mL) was treated at room temperature with N,N-diisopropylethylamine (206 μL, 1.18 mmol) and O-(benzotriazol-1-yl)-N,N,N′,N′-tetramethyluronium tetrafluoroborate (237 mg, 0.738 mmol). After 80 minutes the reaction mixture was concentrated in vacuo. Crushed ice was added to the residue and the formed solid material was filtered, washed with water, dried, and subjected to reversed phase preparative HPLC (20→50% acetonitrile in 0.1 M aqueous ammonium acetate) to give 190 mg (64%) of the title product.

¹H NMR (500 MHz, methanol-d₄ as solvent and internal reference) δ (ppm) 1.59-1.75 (m, 4H), 2.42 (s, 3H), 2.95-3.12 (m, 7H), 3.65-3.75 (m, 4H), 4.05-4.09 (m, 2H), 6.80-6.83 (m, 2H), 7.50 (dd, 1H, J=1.6, 8.6 Hz), 7.58 (s, 1H), 7.73 (d, 1H, J=8.6 Hz), 7.89 (d, 1H, J=1.0 Hz), 7.94 (d, 1H, J=6.8 Hz).

HRMS (ESI+) calc. [M+H]⁺ 502.1674, found 502.1610.

### EXAMPLE 12

### [4-(3-Chloro-1H-indole-6-sulfonyl)-piperazin-1-yl]-(3′-chloro-3,4,5,6-tetrahydro-2H-[1,4′]bipyridinyl-4-yl)-methanone

Treating 4-(1H-indole-6-sulfonyl)-piperazin-1-yl]-(3,4,5,6-tetrahydro-2H-[1,4′]bipyridinyl-4-yl)-methanone (42 mg, 0.093 mmol) with N-chlorosuccinimide as described in example 13, but instead using 2.4 equivalents of N-chlorosuccinimide, also gave 8 mg (16%) of the dichlorinated compound, the title product.

¹H NMR (500 MHz, methanol-d₄ as solvent and internal reference) δ (ppm) 1.72-1.77 (m, 4H), 2.81-2.88 (m, 3H), 3.02-3.11 (m, 4H), 3.66-3.74, 6H), 7.02 (d, 1H, J=5.7 Hz), 7.53 (dd, 1H, J=1.6, 8.3 Hz), 7.59 (s, 1H), 7.75 (d, 1H, J=8.3 Hz), 7.91-7.92 (m, 1H), 8.23 (d, 1H, J=5.5 Hz), 8.31 (broad s, 1H).

HRMS (ESI+) calc. [M+H]⁺ 522.1126, found 522.1133.

### EXAMPLE 13

### [4-(3-Chloro-1H-indole-6-sulfonyl)-piperazin-1-yl]-(3,4,5,6-tetrahydro-2H-[1,4′]bipyridinyl-4-yl)-methanone

A solution of 4-(1H-indole-6-sulfonyl)-piperazin-1-yl]-(3,4,5,6-tetrahydro-2H-[1,4′]bipyridinyl-4-yl)-methanone (135 mg, 0.298 mmol) in dry N,N-dimethyl-formamide (4 mL) was treated at room temperature with N-chlorosuccinimide (40 mg, 0.30 mmol). After 2 hours additional N-chlorosuccinimide was added (10 mg, 0.075 mmol) and reaction mixture was stirred for a further 50 minutes. Crushed ice was then added and the reaction mixture was then concentrated in vacuo. The residue was subjected to reversed phase preparative HPLC (20→50% acetonitrile in 0.1 M aqueous ammonium acetate) to give 77 mg (53%) of the title product.

¹H NMR (500 MHz, methanol-d₄ as solvent and internal reference) δ (ppm) 1.61-1.76 (m, 4H), 2.91-3.00 (m, 1H), 3.01-3.09 (m, 6H), 3.67-3.75 (m, 4H), 4.02-4.07 (m, 2H), 6.91 (broad s, 2H), 7.53 (dd, 1H, J=1.6, 8.3 Hz), 7.60 (s, 1H), 7.75 (d, 1H, J=8.6 Hz), 7.91-7.92 (m, 1H), 8.09 (broad s, 2H).

HRMS (ESI+) calc. [M+H]⁺ 488.1517, found 488.1529.

### EXAMPLE 14

### [4-(1H-Indole-6-sulfonyl)-piperazin-1-yl]-(3,4,5,6-tetrahydro-2H-[1,4′]bipyridinyl-4-yl)-methanone

To a suspension of 6-piperazine-1-sulfonyl)-1H-indole hydrochloride (188 mg, 0.623 mmol) and 3,4,5,6-tetrahydro-2H-[1,4′]bipyridinyl-4-carboxylic acid (139 mg, 0.673 mmol) in dry N,N-dimethylformamide (3 mL) at room temperature was added NAN-diisopropylethylamine (289 μL, 1.66 mmol) and O-(benzotriazol-yl)-N,N,N′,N′-tetramethyluronium tetrafluoroborate (259 mg, 0.807 mmol). The reaction mixture was stirred at 40° C. for 50 minutes. Crushed ice was then added and the reaction mixture was then concentrated in vacuo. The residue was subjected to reversed phase preparative HPLC (25×45% acetonitrile in 0.1 M aqueous ammonium acetate) to give 135 mg (48%) of the title product.

¹H NMR (300 MHz, dimethyl sulfoxide-d₆ as solvent and internal reference) δ (ppm) 1.35-1.58 (m, 4H), 2.76-2.89 (m, 7H), 3.49-3.64 (m, 4H), 3.81-2.89 (m, 2H), 6.60 (broad s, 1H), 6.75 (d, 2H, J=5.0 Hz), 7.31 (dd, 1H, J=1.6, 8.3 Hz), 7.67 (t, 1H, J=2.8 Hz), 7.76 (d, 1H, J=8.4 Hz), 7.80 (broad s, 1H), 8.08 (broad s, 2H), 11.64 (s, 1H).

HRMS (ESI+) calc. [M+H]⁺ 454.1907, found 454.1929.

### EXAMPLE 15

### 6-{4-[4-(3-Chloro-1H-indole-6-sulfonyl)-3,4-dihydro-2H-pyrazine-1-carbonyl]piperidin-1-yl}-2-methyl-2H-pyridazin-3-one

To 6-{4-[4-(3-chloro-1H-indole-6-sulfonyl)-3-hydroxy-piperazine-1-carbonyl]-piperidin-1-yl}-2-methyl-2H-pyridazin-3-one, i.e. the title product of Example 3, (10 mg, 0.020 mmol) dissolved in 2 n3L methanol was added one drop of concentrated hydrochloric acid. The reaction was run for 1 hour at room temperature. The mixture was concentrated in vacuo to give 9 mg (93% yield) of the title product.

¹H NMR (400 MHz; acetonitrile-d₃ as solvent and internal reference) δ (ppm) 1.63-1.81 (m, 4H), 2.75 (m, 1H), 3.02 (m, 2H), 3.35 (m, 1H), 3.42 (m, 1H), 3.52 (m, 1H), 3.54 (m, 1H), 3.74 (s, 3H), 3.92 (broad d, 2H, J=13.1 Hz), 6.12 (d, 0.7H, J=6.7 Hz, rotamer), 6.23 (d, 0.3H, J=6.7 Hz, rotamer), 6.35 (d, 0.7H, J=6.9 Hz, rotamer), 6.66 (d, 0.3H, J=6.9 Hz, rotamer), 7.51-7.58 (m, 2H), 7.63 (m, 2H), 7.73 (broad d, 1H, J=9.3 Hz), 7.98 (s, 1H).

HRMS (ESI+) calc. [M+M]+517.1425, found 517.1441.

### EXAMPLE 16

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide

4-(3-Chloro-1H-indole-6-sulfonyl) 1-[1-(1-methyl-6-oxo-1,6-dihydro-pyridazin-3-yl)-piperidin-4-ylmethyl]-6-oxo-piperazine-2-carboxylic acid, i.e. the title product of Example 1, (50 mg, 0.09 mmol), 2-(7-aza-1H-benzotriazole-1-yl)-1,1,3,3-tetra-methyluronium hexafluorophosphate (37 mg, 0.10 mmol) and dimethylamine hydro-chloride (22 mg, 0.27 mmol) was dissolved in 2 mL dry N,N-dimethylformamide before N,N-diisopropylethylamine (0.077 mL, 0.44 mmol) was added. The reaction mixture was stirred over night at room temperature. Additional N,N-diisopropylethylamine (1 eq.), dimethylamine hydrochloride (1 eq.) and 2-(7-aza-1H-benzotriazole-1-yl)-1,1,3,3-tetramethyluronium hexafluorophosphate (1 eq) was added followed by benzotriazol-yl-oxytri-pyrrolidino phosphonium hexafluorophosphate (46 mg, 0.090 mmol). After 2 hours the mixture was purified by preparative HPLC using a gradient of acetonitrile/5% acetonitrile in water buffer containing 0.1 M ammonium acetate to give the product and a by-product from benzotriazol-1-yl-oxytri-pyrrolidinophos-phonium hexafluorophosphate. The crude was dissolved in ethyl acetate and washed three times with 1 M hydrochloric acid and once with water, dried over sodium sulfate, filtered and evaporated in vacuo to give 7.5 mg (14% yield) of the title product as a white powder.

¹H NMR (400 MHz, methanol-d₄ as solvent and internal reference) δ (ppm) 1.18 (m, 2H), 1.52 (broad d, 1H, J=13.0 Hz), 1.65 (broad d, 1H, J=13.0 Hz), 1.73 (m, 1H), 2.49 (m, 1H), 2.63 (m, 2H), 2.85 (s, 3H), 3.06 (s, 3H), 3.16 (m, 1H), 3.49 (d, 1H, J=16.7 Hz), 3.58 (s, 3H), 3.72 (m, 1H), 3.78-3.93 (m, 3H), 4.01 (d, 1H, J=16.7 Hz), 4.66 (m, 1H), 6.79 (d, 1H, J=10.0 Hz), 7.38 (d, 1H, J=10.0 Hz), 7.51 (m, 1H), 7.57 (s, 1H), 7.73 (d, 1H, J=8.3 Hz), 7.9 (s, 1H).

HRMS (ESI+) calc. [M+H]⁺ 590.1953, found 590.1965.

### EXAMPLE 17

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (53 mg, 0.09 mmol), triethylamine (0.06 mL, 0.44 mmol) and ethylamine hydrochloride (14 mg, 0.18 mmol) was dissolved in 1.8 mL dry N,N-dimethylformamide. Benzotriazol-yl-oxytripyrrolidinophosphonium hexafluorophosphate (69 mg, 0.13 mmol) was added in one portion. The reaction was stirred for two hours at room temperature. The mixture was purified by preparative HPLC using a gradient of acetonitrile/5% acetonitrile in water buffer containing 0.1 M ammonium acetate to give the product and a by-product from benzotriazole-1-yl-oxy-tris-pyrrolidino-phosphonium hexafluorophosphate. The crude was further purified by flash chromatography on silica gel using dichloromethane/methanol (95:5) as eluent to give the product containing a small amount of byproduct. The crude was dissolved in ethyl acetate and washed with 1 M hydrochloric acid and water, dried over sodium sulfate, filtered and evaporated in vacuo to give pure title product, 25 mg, (45% yield) as a white powder.

¹H NMR (400 MHz, methanol-d₄ as solvent and internal reference) δ (ppm) 1.11 (t, 3H, J=7.2 Hz), 1.1-1.3 (m, 2H), 1.49 (broad d, 1H, J=13.3 Hz), 1.61 (broad d, 1H, J=13.3 Hz), 1.75 (m, 1H), 2.49-2.66 (m, 3H), 3.13 (m, 1H), 3.20 (q, 2H, J=7.2 Hz), 3.46 (d, 1H, J=16.1 Hz), 3.57 (s, 3H), 3.76-3.93 (m, 4H), 4.0 (d, 1H, J=16.1 Hz), 4.09 (m, 1H), 6.79 (d, 1H, J=9.3 Hz), 7.38 (d, 1H, J=9.3 Hz), 7.51 (m, 1H), 7.57 (s, 1H), 7.73 (d, 1H, J=8.6 Hz), 7.90 (s, 1H).

HRMS (ESI+) calc. [M+H]⁺ 590.1953, found 590.1959.

### EXAMPLE 18

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (50 mg, 0.090 mmol), triethylamine (0.10 μL, 0.72 mmol) and ethanol amine (11 mg, 0.18 mmol) was dissolved in 1.8 mL dry N,N-dimethylformamide. Benzotriazol-1-yl-oxytri-pyrrolidinophosphonium hexafluorophosphate (69 mg, 0.13 mmol) was added in one portion. The reaction was stirred over night at room temperature. The mixture was purified by preparative HPLC using a gradient of acetonitrile/5% acetonitrile in water buffer containing 0.1 M ammonium acetate to give 42 mg (78% yield) of the desired title product after freeze drying over night.

¹H NMR (300 MHz, acetic acid-d₄ as solvent and internal reference) δ (ppm) 1.24 (m, 2H), 1.48-1.68 (m, 2H), 1.89 (m, 1H), 2.67 (m, 3H), 3.12 (m, 1H), 3.49 (t, 2H, J=5.2 Hz), 3.58 (d, 1H, J=16.7 Hz), 3.66 (s, 3H), 3.79 (t, 2H, J=5.2 Hz), 3.84-4.0 (m, 3H), 4.10 (m, 1H), 4.19 (d, 1H, J=16.7 Hz), 4.36 (m, 1H), 7.10 (d, 1H, J=9.4 Hz), 7.34 (d, 1H, J=9.4 Hz), 7.55 (m, 2H), 7.75 (d, 1H, J=7.7 Hz), 7.99 (m, 1H).

HRMS (ESI+) calc. [M+H]⁺ 606.1901, found 606.193.

### EXAMPLE 19

### 6-{4-[4-(3-Chloro-1H-indole-6-sulfonyl)-2-(morpholine 4-carbonyl)-6-oxo-piperazin-1-ylmethyl]-piperidin-1-yl}-2-methyl-2H-pyridazin-3-one

### i) 6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one and ii) 6-{4-[(S)-4-(3-Chloro-1H-indole-6-sulfonyl)-2-(morpholine 4-carbonyl)-6-oxo-piperazin-1-ylmethyl]-piperidin-1-yl}-2-methyl-2H-pyridazin-3-one

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (78 mg, 0.14 mmol) and morpholine (0.050 mL, 0.57 mmol) was dissolved in 1.5 mL dry N,N-dimethylformamide, 2-(1H-benzotriazole-1-yl)-1,1,3,3-tetramethyl-uronium tetrafluoroborate (54 mg, 0.17 mmol) was added in one portion. The reaction was stirred for 4 hours at room temperature. More 2-(1H-benzotriazole-1-yl)-1,1,3,3-tetramethyluronium tetrafluoroborate (25 mg, 0.080 mmol) was added and the mixture was stirred for 1 hour. The crude mixture was purified by preparative HPLC using acetonitrile/5% acetonitrile in water buffer containing 0.1 M ammonium acetate to give 60 mg (68% yield) of the title compound as a light yellow powder after evaporation of solvent and freeze drying over night.

¹H NMR (400 MHz, methano-d₄ as solvent and internal reference) δ (ppm) 1.19 (m, 2H), 1.54 (broad d, 1H, J=12.9 Hz), 1.66 (broad d, 1H, J=12.9 Hz), 1.75 (m, 1H), 2.51 (m, 1H), 2.63 (m, 2H), 3.07 (m, 1H), 3.42 (m, 2H), 3.49-3.94 (m, 14H), 4.04 (d, 1H, J=16.7 Hz), 4.64 (m, 1H), 6.79 (d, 1H, J=9.8), 7.38 (d, 1H, J=−9.8 Hz), 7.51 (m, 1H), 7.57 (s, 1H), 7.73 (d, 1H, J=8.2 Hz), 7.90 (s, 1H).

The enantiomers i) and ii) were separated by preparative chiral chromatography.
i) HRMS (ESI+) calc. [M+H]⁺ 632.2058, found 632.2092.
ii) HRMS (ESI+) calc. [M+H]⁺ 632.2058, found 632.2092.

### EXAMPLE 20

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide

### i) (R)-4-(3-Chloro-1H-indole-6-sulfonyl-1-[1-(1-methyl-6-oxo-1,6-dihydro-pyridazin-3-yl)-piperidin-4-ylmethyl]-6-oxo-piperazine-2-carboxylic acid isopropylamide and ii) (S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid isopropylamide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, the title product of Example 1 (54 mg, 0.096 mmol) was dissolved in 1 ml dry N,N-dimethylformamide, diisopropyl-ethylamine (0.031 mL, 0.18 mmol) and 2-(1H-benzotriazole-1-yl)-1,1,3,3-tetramethyl-uronium tetrafluoroborate (34 mg, 0.11 mmol) was added. The mixture was stirred for 5 minutes at room temperature before N,N-diisopropylamine (0.030 mL, 0.35 mmol) was added. The reaction mixture was stirred over night. More N,N-diisopropylethylamine (0.10 μL, 0.57 mmol), 2-(1H-benzotriazole-1-yl)-1,1,3,3-tetramethyluronium tetra-fluoroborate (31 mg, 0.096 mmol) and isopropylamine (0.10 mL, 1.2 mmol) was added. After 2 days part of the solvent was evaporated in vacuo and the crude was purified by preparative HPLC using a gradient of acetonitrile/5% acetonitrile in water phase containing 0.1 M ammonium acetate to give 31 mg (53% yield) of the desired title compound as a white powder after evaporation of solvent and freeze drying over night.

¹H NMR (400 MHz, methanol-d₄ as solvent and internal reference) δ (ppm) 1.11-1.24 (m, 8H), 1.49 (broad d, 1H, J=12.8 Hz), 1.62 (broad d, 1H, J=12.8 Hz), 1.74 (m, 1H), 2.59 (m, 3H), 3.15 (m, 1H), 3.46 (d, 1H, J=16.1 Hz), 3.58 (s, 3H), 3.73-3.87 (m, 4H), 3.93-4.01 (m, 2H), 4.08 (m, 1H), 6.79 (d, 1H, J=9.8 Hz), 7.38 (d, 1H, J=9.8 Hz), 7.51 (m, 1H), 7.57 (s, 1H), 7.73 (d, 1H, J=8.5 Hz), 7.90 (s, 1H).

The enantiomers i) and ii) were separated by preparative chiral chromatography.
i) HRMS (ESI+) calc. [M+H]⁺ 590.1953, found 590.1964.

### EXAMPLE 21

### 6-{4-[2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one

### i) 6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one and ii) 6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, the title product of Example 1 (62 mg, 0.11 mmol), was dissolved in 1.1 mL N,N-dimethylformamide, N,N-diisopropylethylamine (0.038 mL, 0.22 mmol) and 2-(1H-benzotriazole-1-yl)-1,1,3,3-tetramethyluronium tetrafluoroborate (39 mg, 0.12 mmol) was added. The mixture was stirred for 5 minutes before azetidine (0.03 mL, 0.44 mmol) was added. The reaction mixture was stirred over night. More N,N-diisopropylethylamine (0.1 mL, 0.57 mmol), 2-(1H-benzo triazole-1-yl)-1,1,3,3-tetramethyluronium tetrafluoroborate (40 mg, 0.12 mmol) and azetidine (0.03 mL, 0.44 mmol) was added. After 2 days, part of the solvent was evaporated in vacuo and the crude was purified by preparative HPLC using a gradient of acetonitrile/5% acetonitrile in water phase containing 0.1 M ammonium acetate to give 39 mg (58% yield) of the desired title compound as a light yellow powder after evaporation of solvent and freeze drying over night.

¹H NMR (400 MHz, methanol-d₄ as solvent and internal reference) δ (ppm) 1.17 (m, 2H), 1.50 (broad d, 1H, J=12.2 Hz), 1.62 (broad d, 1H, J=12.2 Hz), 1.73 (m, 1H), 2.25 (m, 2H), 2.59 (m, 3H), 3.18 (m, 1H), 3.54 (d, 1H, J=16.4 Hz), 3.58 (s, 3H), 3.72-3.86 (m, 5H), 4.00 (m, 2H), 4.14 (m, 1H), 4.21-4.31 (m, 2H), 6.80 (d, 1H, J=10.1 Hz), 7.38 (d, 1H, J=10.1 Hz), 7.55 (m, 1H), 7.58 (s, 1H), 7.75 (d, 1H, 8.8 Hz), 7.93 (s, 1H).

The enantiomers i) and ii) were isolated by preparative chiral chromatography.
i) HRMS (ESI+) calc. [M+H]⁺ 602.1953, found 602.1948.
ii) HRMS (ESI+) calc. [M+H]⁺ 602.1953, found 602.1958.

### EXAMPLE 22

### 6-{4-[4-(3-Chloro-1H-indole-6-sulfonyl)-2-hydroxymethyl-6-oxo-piperazin-1-ylmethyl]-piperidine-1-yl}-2-methyl-2H-pyridazin-3-one

### A) 6-{4-[4-(1-Benzenesulfonyl-3-chloro-1H-indole-6-sulfonyl)-2-hydroxymethyl-6-oxo-piperazin-1-ylmethyl]-piperidin-1-yl}-2-methyl-2H-pyridazin-3-one

4-(1-Benzenesulfonyl-3-chloro-1H-indole-6-sulfonyl)-1[1-(1-methyl-6-oxo-1,6-dihydro-pyridazine-3-yl)-piperidin-4-ylmethyl]-6-oxo-piperazine-2 carboxylic acid, the title product of Example 1 (30 mg, 0.040 mmol) was dissolved in tetrahydrofuran (5 ml) together with triethylamine (5 mg, 0.05 mmol). The reaction mixture was cooled on an ice/salt bath to −18° C. and isobutyl chloroformate (6 mg, 0.05 mmol) was added. After 30 minutes the formed precipitate was filtered off and the reaction mixture was cooled again to −18° C. Sodium borohydride (5 mg, 0.13 mmol) was added and a few drops of water. When the foaming was over another 2 mL of water was added and the reaction mixture was allowed to stand at ambient temperature for 1 hour. Water was added, tetrahydrofuran was removed in vacuo and the remaining water phase was extracted three times with dichloromethane. The combined organic phase was washed with water and brine, dried with sodium sulfate and after filtration the solvent was evaporated in vacuo to give 30 mg of the sub-title compound which was used without further purification in the next step.

### B)

The intermediate was dissolved in tetrahydrofuran (2 mL) and lithium hydroxide (2 mg, 0.09 mmol) dissolved in water (1 mL) was added. The reaction mixture was allowed to stand at ambient temperature for 2 hours whereupon the pH was adjusted to 5-6 by addition of 0.1 M hydrochloric acid. Water (20 mL) was added, tetrahydrofuran was removed in vacuo and the remaining water phase was extracted three times with dichloromethane (20 mL). The combined organic phase was washed with water and brine, dried with sodium sulfate and the solvent evaporated in vacuo. The residue was purified by HPLC (Kromasil C8) using a gradient of acetonitrile (20-70% in water containing 0.1 M ammonium acetate to give 4.5 mg of the title compound after evaporation and freeze drying.

¹H NMR (400 MHz, methanol-d₄ as solvent and internal reference) δ (ppm): 1.04-1.20 (m, 1H), 1.19-1.30 (m, 1H), 1.45-1.52 (broad d, 1H), 1.58-1.65 (broad d, 1H), 1.81-1.90 (m, 1H), 2.62 (q, 2H, J=12 Hz), 2.80-2.91 (m, 2H), 3.38 (d, 1H, J=17.6 Hz), 3.46-3.52 (m, 1H), 3.59 (s, 3H), 3.67-3.76 (m, 2H), 3.77-3.89 (m, 3H), 3.90-3.98 (m, 2H), 6.81 (d, 1H, J=10 Hz), 7.38 (d, 1H, J=10 Hz), 7.55 (d, 1H, J=8.8 Hz), 7.58 (s, 1H), 7.76 (d, 1H, J=8.8 Hz), 7.94 (s, 1H).

HRMS (ESI+) calc. [M+H]⁺ 549.1687, found 549.1686.

### EXAMPLE 23

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide

### i) (R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide and ii) (S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide

4-(3-Chloro-1H-indole-6-sulfonyl) 1-[1-(1-methyl-6-oxo-1,6-dihydro-pyridazin-3-yl)-piperidin-4-ylmethyl]-6-oxo-piperazine-2-carboxylic acid, the title product of Example 1, (40 mg, 0.071 mmol) was dissolved in 1 mL dry N,N-dimethylformamide and 2-(1H-benzotriazole-1-yl)-1,1,3,3-tetramethyluronium tetrafluoroborate (91 mg, 0.28 mmol) was added. The mixture was stirred for 5 minutes at room temperature before 2-methoxy-ethylamine (0.031 ml, 0.36 mmol) was added. The reaction mixture was stirred for 1 hour. The crude material was purified by preparative HPLC using a gradient of acetonitrile/5% acetonitrile in water phase containing 0.1 M ammonium acetate to give 40 mg (91% yield) of the desired title compound as a white powder after evaporation of solvent and freeze drying over night.

¹H NMR (500 MHz, methanol-d₄ as solvent and internal reference) δ (ppm) 1.10-1.27 (m, 2H), 1.50 (broad d, 1H, J=13 Hz), 1.63 (broad d, 1H, J=13 Hz), 1.72-1.82 (m, 1H), 2.55-2.69 (m, 3H), 3.14-3.20 (m, 1H), 3.35 (s, 3H), 3.36-3.52 (m, 5H), 3.59 (s, 3H), 3.77-3.92 (m, 4H), 4.01 (d, 1H, J=17 Hz), 4.14-4.18 (m, 1H), 6.81 (d, 1H, J=10 Hz), 7.40 (d, 1H, J=10 Hz), 7.51-7.55 (m, 1H), 7.59 (s, 1H), 7.75 (d, 1H, J=9 Hz), 7.92 (s, 1H).

The enantiomers i) and ii) were isolated by preparative chiral chromatography.
i) HRMS (ESI+) calc. [M+H]⁺ 620.2058, found 602.2055.
ii) HRMS (ESI+) calc. [M+H]⁺ 620.2058, found 602.2056.

### EXAMPLE 24

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid tert-butyl ester

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (50 mg, 0.089 mmol) was suspended in dry toluene (1.5 ml). N,N-dimethylformamide di-tert-butyl acetal (72 mg, 0.36 mmol) was added dropwise before the reaction mixture was heated at 85° C. (oil bath temperature). One equivalent of N,N-dimethylformamide di-tert-butyl acetal was added dropwise. The reaction mixture was stirred for an additional hour. This procedure was repeated twice. The reaction mixture was cooled and concentrated under reduced pressure before purification by prep-HPLC using a gradient of acetonitrile/5% acetonitrile in a water phase containing 0.1 M ammonium acetate to give 15 mg (27% yield) of the desired title compound as a white powder after evaporation of solvent and freeze drying over night.

¹H NMR (500 MHz, dimethyl sulfoxide-d₆ as solvent and internal reference) δ (ppm) 0.96-1.17 (m, 2H), 1.43-1.48 (m, 10H), 1.57 (broad d, 1H, J=14 Hz), 1.62-1.72 (m, 1H), 2.46-2.58 (m, 3H), 2.90 (dd, 1H, J=3, 12 Hz), 3.22 (d, 1H, J=16 Hz), 3.44 (s, 3H), 3.63-3.82 (m, 4H), 4.00 (d, 1H, J=12 Hz), 4.24-4.27 (m, 1H), 6.75 (d, 1H, J=10 Hz), 7.41 (d, 1H, J=10 Hz), 7.48 (dd, 1H, J=2, 8 Hz), 7.72 (d, 1H, J=8 Hz), 7.87 (d, 1H, J=1 Hz), 7.88 (s, 1H).

HRMS (ESI+) calc. [M+H]⁺ 619.2106, found 619.207.

### EXAMPLE 25

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester

To a reaction vial containing 4-(3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (12 mg, 0.021 mmol) was added hydrochloric acid-saturated ethanol. The reaction vial was equipped with a septum and the reaction mixture was heated at 70° C. for 90 min. The reaction mixture was evaporated to dryness under reduced pressure before the crude was dissolved in dimethyl sulfoxide and purified by preparative HPLC using a gradient of acetonitrile/5% acetonitrile in a water phase containing 0.1 M ammonium acetate to give 12 mg (95% yield) of the desired title compound as a white powder after evaporation of solvent and freeze drying over night.

¹H NMR (500 MHz, acetonitrile-d₃ as solvent and internal reference) δ (ppm) 1.08 (dq, 1H, J=4, 12 Hz), 1.18 (dq, 1H J=4, 12 Hz), 1.25 (t, 3H, J=7 Hz), 1.49 (broad d, 1H, J=13 Hz), 1.59 (broad d, 1H, J=13 Hz), 1.63-1.73 (m, 1 M), 2.50-2.59 (m, 3H), 2.94 (dd, 1H, J=3, 12 Hz), 3.32 (d, 1H, J=16 Hz), 3.48 (s, 3H), 3.65-3.76 (m, 2 H), 3.81 (dd, 1H, J=8, 14 Hz), 3.93 (d, 1H, J=16 Hz), 4.10 (din, 1H, J=12 Hz), 4.12-4.24 (m, 3H), 6.67 (d, 1H, J=10 Hz), 7.18 (d, 1H, J=10 Hz), 7.52 (dd, 1H, J=1, 8 Hz), 7.57 (d, 1H, J=3 Hz), 7.74 (d, 1H, J=8 Hz), 7.95 (s, 1H), 9.96 (s, 1 NH).

HRMS (ESI+) calc. [M+H]⁺ 591.1793, found 591.1782.

### EXAMPLE 26

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropyl ester

To a reaction vial containing 4-(3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (180 mg, 0.32 mmol) was added hydrochloric acid-saturated propan2-ol. The reaction vial was equipped with a septum and the reaction mixture was heated at 85° C. for 2.5 h. The reaction mixture was evaporated to dryness under reduced pressure before the crude was dissolved in dimethyl sulfoxide and purified by preparative HPLC using a gradient of acetonitrile/5% acetonitrile in a water phase containing 0.1 M ammonium acetate to give 144 mg (74% yield) of the desired title compound as a white powder after evaporation of solvent and freeze drying over night.

¹H NMR (500 MHz, dimethyl sulfoxide-d₆ as solvent and internal reference) (ppm) 1.00 (dq, 1H, J=4, 12 Hz), 1.12 (dq, 1H J=4, 12 Hz), 1.24 (dd, 6H, J=2, 6 Hz), 1.46 (broad d, 1H, J=12 Hz), 1.57 (broad d, 1H, J=12 Hz), 1.64-1.74 (m, 1H), 2.48-2.59 (m, 3H), 2.95 (dd, 1H, J=3, 12 Hz), 3.25 (d, 1H, J=16 Hz), 3.44 (s, 3H), 3.65-3.77 (m, 3H), 3.79 (dd, 1H, J=16 Hz), 4.00 (d, 1H, J=12 Hz), 4.33-4.36 (m, 1H), 4.98 (sept., 1H, J=6 Hz), 6.75 (d, 1H, J=10 Hz), 7.41 (d, 1H, J=10 Hz), 7.47 (dd, 1H, J=2, 8 Hz), 7.72 (d, 1H, J=8 Hz), 7.87 (d, 1H, J=1 Hz), 7.88 (s, 1H).

HRMS (ESI+) calc. [M+H]⁺ 605.1949, found 605.1946.

1. A compound of formula (I) 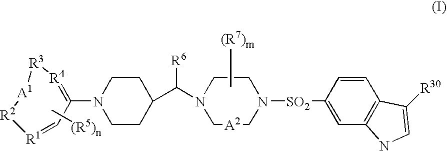
wherein
R¹, R², R³ and R⁴ are independently selected from carbon and nitrogen, and where at least one of R¹, R², R³ and R⁴ is nitrogen;
A¹ is a single bond or a double bond;
n is 0, 1, 2 or 3;
each R⁵ is independently selected from hydrogen, halogen, C₁₋₃alkyl, oxo, oxy, oxido and thioxo;
R⁶ is hydrogen or oxo;
m is 0, 1, 2 or 3;
A² is a single bond or a double bond;
each R⁷ is independently selected from hydrogen, hydroxy, oxo, C₁₋₅alkyl, carboxy, cyano, tetrazolyl, N—C₁₋₅ alkyltetrazolyl, oxazolyl, C₁₋₅ oxazolyl, isoxazolyl, C₁₋₅ isoxazolyl, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, carbamoylC₁₋₄alkyl, C₁₋₅alkylcarbamoylC₁₋₄alkyl, di(C₁₋₅alkyl)carbamoylC₁₋₄alkyl, hydroxyC₁₋₅alkylcarbamoyl, C₁₋₅alkoxyC₁₋₅alkylcarbamoyl, hydroxyC₁₋₅alkylcarbamoylC₁₋₄alkyl, C₁₋₅alkoxyC₁₋₅alkylcarbamoylC₁₋₄alkyl, —CONR⁸(CH₂)ₓS(O)ₚR⁹, —CONH(CH₂)_{q}NR¹⁰R¹¹, —C₁₋₅alkyl-Y¹, —COOCHR¹⁷R¹⁸ and —CONR¹⁷R¹⁸:
wherein x represents an integer 0 to 4;
p is 0, 1 or 2;
q represents an integer 2 to 4;
R⁸ represents hydrogen or C₁₋₃alkyl;
R⁹ represents C₁₋₅alkyl or phenyl; or
R⁸ and R⁹ may together form a C₁₋₅alkylene group;
R¹⁰ and R¹¹ independently represent hydrogen, C₁₋₅alkyl, phenyl, C₁₋₅alkylphenyl, S(O)ₚR⁹, COR¹² or a 5- or 6-membered monocyclic heteroaryl ring containing up to 3 heteroatoms selected from nitrogen, oxygen and sulphur;
R¹² represents hydrogen, C₁₋₅alkyl or phenyl;
Y¹ represents S(O)ₚR⁹, NHS(O)₂R⁹, NHCOR¹³, O(CH₂)ᵣR¹⁴, azetidino, pyrrolidin-1-yl, piperidino, morpholino, thiamorpholino, 1-oxothiamorpholino, 1,1-dioxothiamorpholino, piperazin-1-yl or C₁₋₅alkylamino,
R¹³ represents C₁₋₅alkyl, phenyl or C₁₋₅alkylphenyl;
r represents an integer 1 to 4;
when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;
wherein any phenyl group within R⁷ is independently substituted by 0, 1 or 2 substituents selected from halogeno, trifluoromethyl, cyano, C₁₋₅alkyl and C₁₋₅alkoxy;
R¹⁵ and R¹⁶ independently represent hydrogen or C₁₋₅alkyl;
R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl;
R³⁰ is hydrogen, amino, methyl or halogen;
or a pharmaceutically acceptable salt thereof.

2. A compound according to claim 1 wherein one or two of R¹, R², R³ and R⁴ is/are nitrogen.

3. A compound according to claim 1 wherein at least one of R¹, R² and R³ is nitrogen.

4. A compound according to claim 1 wherein A¹ is a single bond.

5. A compound according to claim 1 wherein A¹ is a double bond.

6. A compound according to claim 1 wherein n is 0, 1 or 2.

7. A compound according to claim 1 where one of R⁵ is oxo.

8. A compound according to claim 7 wherein said R⁵ being oxo is positioned at R² and A¹ is a single bond.

9. A compound according to claim 1 where one of R⁵ is C₁₋₃alkyl.

10. A compound according to claim 1 where one of R⁵ is halogen.

11. A compound according to claim 1 where one of R⁵ is oxido.

12. A compound according to claim 1 where n is 2 or 3, one R⁵ is C₁₋₃alkyl, and the other R⁵ is oxo.

13. A compound according to claim 1 wherein m is 0, 1 or 2.

14. A compound according to claim 1 wherein m is 2 or 3.

15. A compound according to claim 1 wherein R⁶ is hydrogen and one R⁷ is oxo.

16. A compound according to claim 1 where each R⁷ is independently selected from hydrogen, hydroxy, oxo, C₁₋₅alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷R¹⁸ and —CONR¹⁷R¹⁸:
wherein
R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

17. A compound according to claim 1, wherein one R⁷ is oxo, and at least one further R⁷ is selected from hydroxy, oxo, C₁₋₅alkyl, carboxy, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, carbamoylC₁₋₄alkyl, C₁₋₅alkylcarbamoylC₁₋₄alkyl, di(C₁₋₅alkyl)carbamoylC₁₋₄alkyl, hydroxyC₁₋₅alkylcarbamoyl, C₁₋₅alkoxyC₁₋₅alkylcarbamoyl, hydroxyC₁₋₅alkylcarbamoylC₁₋₄alkyl, C₁₋₅alkoxyC₁₋₅alkylcarbamoylC₁₋₄alkyl, —CONR⁸(CH₂)ₓS(O)ₚR⁹, —CONH(CH₂)_{q}NR¹⁰R¹¹, —C₁₋₅alkyl-Y¹, —COOCHR¹⁷R¹⁸ and —CONR¹⁷R¹⁸:
wherein x represents an integer 0 to 4;
p is 0, 1 or 2;
q represents an integer 2 to 4;
R⁸ represents hydrogen or C₁₋₃alkyl;
R⁹ represents C₁₋₅alkyl or phenyl; or
R⁸ and R⁹ may together form a C₁₋₅alkylene group;
R¹⁰ and R¹¹ independently represent hydrogen, C₁₋₅alkyl, phenyl, C₁₋₅alkylphenyl, S(O)ₚR⁹, COR¹² or a 5- or 6-membered monocyclic heteroaryl ring containing up to 3 heteroatoms selected from nitrogen, oxygen and sulphur;
R¹² represents hydrogen, C₁₋₅alkyl, phenyl or C₁₋₅alkylphenyl;
Y¹ represents S(O)ₚR⁹, NHS(O)₂R⁹, NHCOR³, O(CH₂)ᵣR¹⁴ pyrrolidin-1-yl, piperidino, morpholino, thiamorpholino, 1-oxothiamorpholino, 1,1-dioxothiamorpholino or piperazin-1-yl,
R¹³ represents C₁₋₅alkyl, phenyl or C₁₋₅alkylphenyl;
r represents an integer 1 to 4;
when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;
wherein any phenyl group within R⁷ is independently substituted by 0, 1 or 2 substituents selected from halogeno, trifluoromethyl, cyano, C₁₋₅alkyl and C₁₋₅alkoxy;
R¹⁵ and R¹⁶ independently represent hydrogen or C₁₋₅alkyl;
R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

18. A compound according to claim 17, wherein said at least one further R⁷ is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —CONR⁸(CH₂)ₓS(O)ₚR⁹, —CONH(CH₂)_{q}NR¹⁰R¹¹, —C₁₋₅alkyl-Y¹, —COOCHR¹⁷R¹⁸ and —CONR¹⁷R¹⁸:
wherein x represents an integer 0 to 4;
p is 0, 1 or 2;
q represents an integer 2 to 4;
R⁸ represents hydrogen or C₁₋₃alkyl;
R⁹ represents C₁₋₅alkyl or phenyl; or
R⁸ and R⁹ may together form a C₁₋₅alkylene group;
R¹⁰ and R¹¹ independently represent hydrogen, C₁₋₅alkyl, phenyl, C₁₋₅alkylphenyl, S(O)ₚR⁹, COR¹² or a 5- or 6-membered monocyclic heteroaryl ring containing up to 3 heteroatoms selected from nitrogen, oxygen and sulphur;
R¹² represents hydrogen, C₁₋₅alkyl, phenyl or C₁₋₅alkylphenyl;
Y¹ represents S(O)ₚR⁹, NHS(O)₂R⁹, NHCOR³, O(CH₂)ᵣR¹⁴ pyrrolidin-1-yl, piperidino, morpholino, thiamorpholino, 1-oxothiamorpholino, 1,1-dioxothiamorpholino or piperazin-1-yl,
R¹³ represents C₁₋₅alkyl, phenyl or C₁₋₅alkylphenyl;
r represents an integer 1 to 4;
when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;
wherein any phenyl group within R⁷ is independently substituted by 0, 1 or 2 substituents selected from halogeno, trifluoromethyl, cyano, C₁₋₅alkyl and C₁₋₅alkoxy;
R¹⁵ and R¹⁶ independently represent hydrogen or C₁₋₅alkyl;
R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

19. A compound according to claim 17, wherein said at least one further R⁷ is selected from hydroxy, C₁₋₃alkyl, carboxy, hydroxyC₁₋₅alkyl, C₁₋₅alkoxyoxoC₁alkyl, carbamoyl, C₁₋₁₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷R¹⁸ and —CONR¹⁷R¹⁸: R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

20. A compound according to claim 17, wherein said at least one further R⁷ is selected from carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl and C₁₋₅alkoxyC₁₋₅alkylcarbamoyl.

21. A compound according to claim 17, wherein said at least one further R⁷ is selected from —COOCHR¹⁷R¹⁸ and —CONR¹⁷R¹⁸: R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0 or 1 additional hetero oxygen, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₁₅alkoxyoxoC₁₋₆alkyl, and carbamoylC₁₋₅alkyl.

22. A compound according to claim 1 wherein R⁶ is oxo.

23. A compound according to claim 22 wherein each R⁷ is independently selected from hydrogen, hydroxy, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, and C₁₋₅alkoxyC₁₋₅alkylcarbamoyl.

24. A compound according to claim 23 wherein one R⁷ is hydroxy.

25. A compound according to claim 1 wherein m is 0.

26. A compound according to claim 1 wherein A² is a single bond.

27. A compound according to claim 25 wherein A² is a double bond.

28. A compound according to claim 1 wherein R³⁰ is halogen.

29. A compound according to claim 1 which is
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester,
6-{4-[4-(3-Chloro-1H-indole-6-sulfonyl)-3-hydroxy-piperazine-1-carbonyl]-piperidin-1-yl}-2-methyl-2H-pyridazin-3-one,
6-{4-[4-(3-Chloro-1H-indole-6-sulfonyl)-2-hydroxy-piperazine-1-carbonyl]-piperidin-1-yl}-2-methyl-2H-pyridazin-3-one,
6-{4-[4-(3-Chloro-1H-indole-6-sulfonyl)-2-oxo-piperazin-1-ylmethyl]-piperidin-1-yl}-2-methyl-2H-pyridazin-3-one,
4-[4-(3-Chloro-1H-indole-6-sulfonyl)-piperazine-1-carbonyl]-5′-methyl-3,4,5,6-tetrahydro-2H,1′H-[1,3′]bipyridinyl-6′-one,
5-{4-[4-(3-Chloro-1H-indole-6-sulfonyl)-piperazine-1-carbonyl]-piperidin-1-yl}-3-methyl-1H-pyrazin-2-one,
6-{4-[4-(3-Chloro-1H-indole-6-sulfonyl)-piperazine-1-carbonyl]-piperidin-1-yl}-2-methyl-2H-pyridazin-3-one,
6-{4-[4-(1H-Indole-6-sulfonyl)-piperazine-1-carbonyl]-piperidin-1-yl}-2-methyl-2H-pyridazin-3-one,
6-{4-[4-(3-Chloro-1H-indole-6-sulfonyl)-piperazine-1-carbonyl]-piperidin-1-yl}-2H-pyridazin-3-one,
[4-(3-Chloro-1H-indole-6-sulfonyl)-piperazin-1-yl]-(1′-oxy-3,4,5,6-tetrahydro-2H-[1,4′]bipyridinyl-4-yl)-methanone,
[4-(3-Chloro-1H-indole-6-sulfonyl)-piperazin-1-yl]-(2′-methyl-3,4,5,6-tetrahydro-2H-[1,4′]bipyridinyl-4-yl)-methanone,
[4-(3-Chloro-1H-indole-6-sulfonyl)-piperazin-1-yl]-(3′-chloro-3,4,5,6-tetrahydro-2H-[1,4′]bipyridinyl-4-yl)-methanone,
[4-(3-Chloro-1H-indole-6-sulfonyl)-piperazin-1-yl]-(3,4,5,6-tetrahydro-2H-[1,4′]bipyridinyl-4-yl)-methanone,
[4-(1H-Indole-6-sulfonyl)-piperazin-1-yl]-(3,4,5,6-tetrahydro-2H-[1,4′]bipyridinyl-4-yl)-methanone,
6-{4-[4-(3-Chloro-1H-indole-6-sulfonyl)-3,4-dihydro-2H-pyrazine-1-carbonyl]piperidin-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide,
6-{4-[4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(S)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid isopropylamide,
6-{4-[2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[4-(3-Chloro-1H-indole-6-sulfonyl)-2-hydroxymethyl-6-oxo-piperazin-1-ylmethyl]-piperidine-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid tert-butyl ester,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester, or
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropyl ester.

30. A process for preparing a compound of formula (I) as defined in claim 1 which process comprises either
(a) reacting an amine of formula (II) 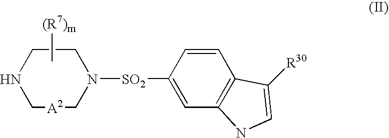
wherein R⁷, R³⁰, A², and m are as defined in claim 1, with a carboxylic acid of the formula (III) 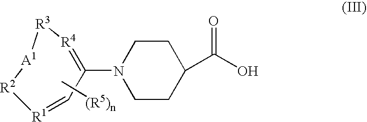
wherein R¹, R², R³, R⁴, R⁵, A¹ and n are as defined in claim 1 or a reactive derivative thereof, or
(b) reacting the compound of formula (V) 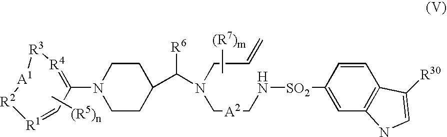
wherein R¹, R², R³, R⁴, R⁵, R⁶, R⁷, R³⁰, A¹, A², m, and n are as defined in claim 1 with an oxidizing agent; or
(c) reacting the compound of formula (VII) 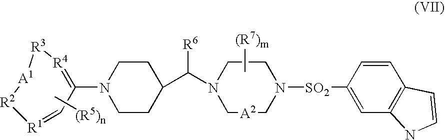
wherein R¹, R², R³, R⁴, R⁵, R⁶, R⁷, A¹, A², m, and n are as defined in claim 1 with the corresponding halogen succinimide; or
(d) carrying out a reaction with the compound of formula (IV) 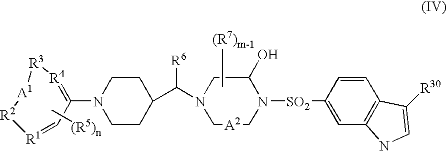
wherein R¹, R², R³, R⁴, R⁵, R⁶, R⁷, R³⁰, A¹, A², m, and n are as defined in claim 1 in acidic conditions; or
(e) where the compound of formula (I) is a compound of formula (IX) 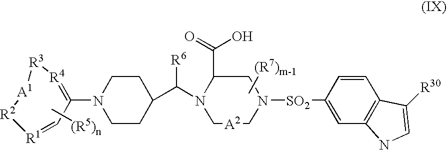
wherein R¹, R², R³, R⁴, R⁵, R⁶, R⁷, R³⁰, A¹, A², m, and n are as defined in claim 1, or a reactive derivative thereof,
conditions are used as described in process (a);
(f) where the compound of formula (I) is an ester derivative of the compound of formula (IX), the compound of formula (IX) are treated in a readily available alcoholic solvent using acid catalysis, and using in the case of hindered alcohols N,N-dimethylformamide dialkyl acetal;
(g) reacting a sulfonyl chloride derivative of formula (X), with or without a protecting group on the indolyl nitrogen, 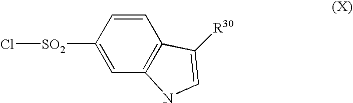
wherein R³⁰ is as defined in claim 1, with an amine of formula (XI) 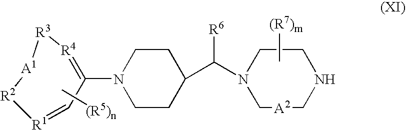
wherein R¹, R², R³, R⁴, R⁵, R⁶, R⁷, A¹, A², m, and n are as defined in claim 1, or a salt thereof;
(h) reacting a carboxylic acid derivative of formula (IX), or a reactive intermediate thereof followed by addition of a reducing agent; or
(i) oxidative cleaving of the exocyclic double bond of formula (XIV) 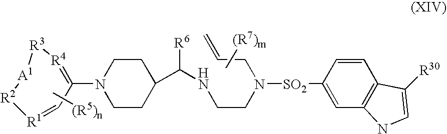
wherein R¹, R², R³, R⁴, R⁵, R⁶, R⁷, R³⁰, A¹, m, and n are as defined in claim 1.

31. (canceled)

32. A pharmaceutical composition comprising a compound of formula (I), or a pharmaceutically-acceptable salt thereof, as defined in any claim from 1 to 29 claim 1, with a pharmaceutically-acceptable diluent or carrier.

33. (canceled)

34. A method of treating a Factor Xa mediated disease or condition in a warm-blooded animal comprising administering an effective amount of a compound of formula (I), as defined in claim 1, or a pharmaceutically-acceptable salt thereof.

35. A combination comprising a compound of formula (I), as defined in claim 1, or a pharmaceutically-acceptable salt thereof, and one or more antithrombotic agent(s) with a different mechanism of action, wherein said antithrombotic agent(s) is selected from: an anticoagulant, a vitamin K antagonist, a synthetic or biotechnological inhibitor of other coagulation factors than FXa an antiplatelet agent; a thromboxane receptor and/or synthetase inhibitor; a fibrinogen receptor antagonist; a prostacyclin mimetic; a phosphodiesterase inhibitor; an ADP-receptor antagonist; and an inhibitor of carboxypeptidase U and an inhibitor of plasminogen activator inhibitor-1 (PAI-1).

36. A combination comprising a compound of formula (I), as defined in claim 1, or a pharmaceutically-acceptable salt thereof, and a thrombolytic agent.

37. A process according to claim 30 wherein:
the oxidizing agent in (b) is sodium periodate/osmium tetroxide or ozone/dimethyl sulfide;
the alcoholic solvent using acid catalysis in (f) is saturation of the solvent by gaseous hydrochloric acid; and
the carboxylic acid derivative of formula (IX), or a reactive intermediate thereof, in (h) is a mixed anhydride formed by reacting (IX) with an alkyl chloroformate in situ, and the reducing agent is sodium borohydride.

38. A combination according to claim 35 wherein:
the anticoagulant is unfractionated heparin, low molecular weight heparin, other heparin derivative, or synthetic heparin derivative;
the synthetic or biotechnological inhibitor of other coagulation factors than FXa is an inhibitor of synthetic thrombin, FVIIa, FXIa, FIXa, or rNAPc2;
the antiplatelet agent is acetylsalicylic acid, ticlopidine, or clopidogrel;
the ADP-receptor antagonist is an antagonist of P2X1, P2Y1, P2Y12, or P2T; and
the carboxypeptidase U is CPU or TAFIa.

39. A combination according to claim 38 wherein the synthetic heparin derivative is fondaparinux.

40. A combination according to claim 36 wherein the thrombolytic agent is selected from a tissue plasminogen activator, streptokinase, urokinase, prourokinase, anisoylated plasminogen-streptokinase activator complex (APSAC), and an animal salivary gland plasminogen activator.
